SEQUENCE LISTING (12 sequences)

SEQ ID NO: 1 - protein, 448 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNK

SEQ ID NO: 2 - protein, 848 aa, synthetic construct
ALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 3 - protein, 448 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNGINVIQPDGSYRSEELNLVIIGPSADIIQFEPKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNK

SEQ ID NO: 4 - protein, 848 aa, synthetic construct
ALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQASVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINAMENNSGWKVSLNYGEIIWTLQDTQEIKQRVVFKYSQMINISDYINRWIFVTITNNRLNNSKIYINGRLIDQKPISNLGNIHASNNIMFKLDGERDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 5 - protein, 448 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNGINVIQPDGSYRSEELNLVIIGPSADIIQFEGKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNK

SEQ ID NO: 6 - protein, 848 aa, synthetic construct
ALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQASVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINAMENNSGWKVSLNYGEIIWTLQDTQEIKQRVVFKYSQMINISDYINRWIFVTITNNRLNNSKIYINGRLIDQKPISNLGNIHASNNIMFKLDGGRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 7 - DNA, 1347 bp, synthetic construct
  source 1..1347: mol_type = other DNA
atgccgtttgtgaacaaacagtttaactataaagatccggtgaacggcgtggatattgcgtatattaaaattccgaacgcgggtcagatgcagccggtgaaagcgtttaaaattcataacaaaatttgggtgattccggaacgcgatacctttaccaacccggaagaaggcgatctgaacccgccaccggaagcgaaacaagtgccggtgagctattatgatagcacctatctgagcaccgataacgaaaaagataactatctgaaaggcgtgaccaaactgtttgaacgcatttatagcaccgatctgggccgcatgctgctgacgagcattgtgcgcggcattccgttctggggcggcagcaccattgataccgaactgaaagtgattgataccaactgcattaacgtgattcagccggatggcagctatcgcagcgaagaactgaacctggtgattattggcccgagcgcggatattattcagtttgaatgcaaaagctttggccatgaagtgctgaacctgacccgcaacggctatggcagcacgcagtatattcgctttagcccggattttacctttggctttgaagaaagcctggaagtggataccaacccgctgctgggcgcgggcaaatttgcgaccgatccggcggtgaccctggcgcatgaactgattcatgcgggccatcgcctgtatggcattgcgattaacccgaaccgcgtgtttaaagtgaacaccaacgcgtattatgaaatgagcggcctggaagtgagctttgaagaactgcgcacctttggcggccatgatgcgaaatttattgatagcctgcaagaaaacgaatttcgcctgtattactataacaaatttaaagatattgcgagcaccctgaacaaagcgaaaagcattgtgggcaccaccgcgagcctgcagtatatgaaaaacgtgtttaaagaaaaatatctgctgagcgaagatacgagcggcaaatttagcgtggataaactgaaatttgataaactgtataaaatgctgaccgaaatttataccgaagataactttgtgaaattttttaaagtgctgaaccgcaagacctatctgaactttgataaagcggtgtttaaaattaacattgtgccgaaagtgaactataccatttatgatggctttaacctgcgcaacaccaacctggcggcgaactttaacggtcagaacaccgaaattaacaacatgaactttaccaaactgaaaaactttaccggcctgtttgaattttataaactgctgtgcgttcgcggcatcattacgagcaaaaccaaaagcctggataaaggctataacaaataa

SEQ ID NO: 8 - DNA, 2550 bp, synthetic construct
  source 1..2550: mol_type = other DNA
atggcgctgaacgatctgtgcattaaagtgaataattgggatctgttttttagcccgagcgaagataactttaccaacgatctgaacaaaggcgaagaaattacgagcgataccaacattgaagcggcggaagagaacattagtctggatctgattcagcagtattatctgacctttaactttgataacgaaccggaaaacattagtattgaaaacctgagcagcgatattattggtcagctggaactgatgccgaacattgaacgctttccgaacggcaaaaaatatgaactggataaatataccatgtttcattatctgcgcgcgcaagaatttgaacatggcaaaagccgcattgcgctgaccaacagcgtgaacgaagcgctgctgaacccgagccgcgtgtataccttttttagcagcgattatgtgaaaaaagtgaacaaagcgaccgaagcggcgatgtttctgggctgggtggaacagctggtgtatgattttaccgatgagacgagcgaagtgagtaccaccgataaaattgcggatattaccattatcattccgtatattggcccggcgctgaacattggcaacatgctgtataaagatgattttgtgggcgcgctgatttttagcggcgcggtgattctgctggaatttattccggaaatcgcgattccggtgctgggcacctttgcgctggtgagctatattgcgaacaaagtgctgaccgtgcagaccattgataacgcgctgagcaaacgcaacgaaaaatgggatgaagtgtataaatatattgtgaccaactggctggcgaaagtgaacacgcagattgatctgattcgcaaaaaaatgaaagaagcgctggaaaaccaagcggaagcgaccaaggcgattattaactatcagtataatcagtataccgaagaggaaaaaaacaacattaactttaacattgatgatctgagcagcaaattaaatgaaagcattaacaaagcgatgatcaacattaacaagtttctgaatcagtgcagcgtgagctatctgatgaacagcatgattccgtatggcgtgaaacgcctggaagattttgatgcgagcctgaaagatgcgctgctgaaatatatttatgataaccgcggcaccctgattggccaagtggatcgcctgaaagataaagttaataacacgctgagcaccgatattccgtttcagctgagcaaatatgtggataatcagcgcctgctgagcacctttaccgaatatattaaaaacattattaacacgagcattctgaacctgcgctatgaaagcaaccatctgattgatctgagccgctatgcgagcaaaattaacattggcagcaaagtgaactttgatccgattgataaaaatcagattcagctgtttaacctggaaagcagcaaaattgaagtgattctgaaaaacgcgattgtgtataacagcatgtatgaaaactttagcacgagcttttggattcgcattccgaaatactttaacagcatcagcctgaacaacgaatataccattattaactgcatggaaaacaacagcggctggaaagtgagcctgaactatggcgaaattatttggaccctgcaagatacccaagaaattaaacagcgcgtggtgtttaaatatagtcagatgattaacattagcgattatattaaccgctggatttttgtgaccattaccaacaaccgtctgaacaacagcaaaatttatattaacggccgcctgattgatcagaaaccgattagcaacctgggcaacattcatgcgagcaacaacattatgtttaaactggatggctgccgcgatacgcatcgctatatctggattaaatattttaatctgttcgacaaagaactgaacgaaaaagaaattaaagatctgtatgataatcagagcaacagcggcattctgaaagatttttggggcgattatctgcagtatgataaaccgtattatatgctgaacctgtatgatccgaacaaatatgtggatgtgaacaacgtgggcattcgcggctatatgtatctgaaaggcccgcgcggcagcgtgatgaccaccaacatttatctgaacagcagcctgtatcgcggcaccaaatttattattaaaaaatatgcgagcggcaacaaagataacattgtgcgcaacaacgatcgcgtgtatattaacgtggttgtgaaaaacaaagaatatcgcctggcgaccaacgcgagccaagcgggcgtggaaaaaattctgagcgcgctggaaattccggatgtgggcaacctgagccaagtggttgtgatgaaaagcaaaaacgatcaaggcattaccaacaagtgcaaaatgaacctgcaagataacaacggcaacgatattggctttattggctttcatcagtttaacaacattgcgaaactggtggcgagcaactggtataaccgtcagattgaacgcagcagccgcaccctgggctgcagctgggaatttattccggttgatgatggctggggcgaacgcccgctgtaa

SEQ ID NO: 9 - DNA, 1347 bp, synthetic construct
  source 1..1347: mol_type = other DNA
atgccgtttgtgaacaaacagtttaactataaagatccggtgaacggcgtggatattgcgtatattaaaattccgaacgcgggtcagatgcagccggtgaaagcgtttaaaattcataacaaaatttgggtgattccggaacgcgatacctttaccaacccggaagaaggcgatctgaacccgccaccggaagcgaaacaagtgccggtgagctattatgatagcacctatctgagcaccgataacgaaaaagataactatctgaaaggcgtgaccaaactgtttgaacgcatttatagcaccgatctgggccgcatgctgctgacgagcattgtgcgcggcattccgttctggggcggcagcaccattgataccgaactgaaagtgattgataccaacggtattaacgtgattcagccggatggcagctatcgcagcgaagaactgaacctggtgattattggcccgagcgcggatattattcagtttgaacctaaaagctttggccatgaagtgctgaacctgacccgcaacggctatggcagcacgcagtatattcgctttagcccggattttacctttggctttgaagaaagcctggaagtggataccaacccgctgctgggcgcgggcaaatttgcgaccgatccggcggtgaccctggcgcatgaactgattcatgcgggccatcgcctgtatggcattgcgattaacccgaaccgcgtgtttaaagtgaacaccaacgcgtattatgaaatgagcggcctggaagtgagctttgaagaactgcgcacctttggcggccatgatgcgaaatttattgatagcctgcaagaaaacgaatttcgcctgtattactataacaaatttaaagatattgcgagcaccctgaacaaagcgaaaagcattgtgggcaccaccgcgagcctgcagtatatgaaaaacgtgtttaaagaaaaatatctgctgagcgaagatacgagcggcaaatttagcgtggataaactgaaatttgataaactgtataaaatgctgaccgaaatttataccgaagataactttgtgaaattttttaaagtgctgaaccgcaagacctatctgaactttgataaagcggtgtttaaaattaacattgtgccgaaagtgaactataccatttatgatggctttaacctgcgcaacaccaacctggcggcgaactttaacggtcagaacaccgaaattaacaacatgaactttaccaaactgaaaaactttaccggcctgtttgaattttataaactgctgtgcgttcgcggcatcattacgagcaaaaccaaaagcctggataaaggctataacaaataa

SEQ ID NO: 10 - DNA, 2550 bp, synthetic construct
  source 1..2550: mol_type = other DNA
atggcgctgaacgatctgtgcattaaagtgaataattgggatctgttttttagcccgagcgaagataactttaccaacgatctgaacaaaggcgaagaaattacgagcgataccaacattgaagcggcggaagagaacattagtctggatctgattcagcagtattatctgacctttaactttgataacgaaccggaaaacattagtattgaaaacctgagcagcgatattattggtcagctggaactgatgccgaacattgaacgctttccgaacggcaaaaaatatgaactggataaatataccatgtttcattatctgcgcgcgcaagaatttgaacatggcaaaagccgcattgcgctgaccaacagcgtgaacgaagcgctgctgaacccgagccgcgtgtataccttttttagcagcgattatgtgaaaaaagtgaacaaagcgaccgaagcggcgatgtttctgggctgggtggaacagctggtgtatgattttaccgatgagacgagcgaagtgagtaccaccgataaaattgcggatattaccattatcattccgtatattggcccggcgctgaacattggcaacatgctgtataaagatgattttgtgggcgcgctgatttttagcggcgcggtgattctgctggaatttattccggaaatcgcgattccggtgctgggcacctttgcgctggtgagctatattgcgaacaaagtgctgaccgtgcagaccattgataacgcgctgagcaaacgcaacgaaaaatgggatgaagtgtataaatatattgtgaccaactggctggcgaaagtgaacacgcagattgatctgattcgcaaaaaaatgaaagaagcgctggaaaaccaagcggaagcgaccaaggcgattattaactatcagtataatcagtataccgaagaggaaaaaaacaacattaactttaacattgatgatctgagcagcaaattaaatgaaagcattaacaaagcgatgatcaacattaacaagtttctgaatcaggcaagcgtgagctatctgatgaacagcatgattccgtatggcgtgaaacgcctggaagattttgatgcgagcctgaaagatgcgctgctgaaatatatttatgataaccgcggcaccctgattggccaagtggatcgcctgaaagataaagttaataacacgctgagcaccgatattccgtttcagctgagcaaatatgtggataatcagcgcctgctgagcacctttaccgaatatattaaaaacattattaacacgagcattctgaacctgcgctatgaaagcaaccatctgattgatctgagccgctatgcgagcaaaattaacattggcagcaaagtgaactttgatccgattgataaaaatcagattcagctgtttaacctggaaagcagcaaaattgaagtgattctgaaaaacgcgattgtgtataacagcatgtatgaaaactttagcacgagcttttggattcgcattccgaaatactttaacagcatcagcctgaacaacgaatataccattattaacgcaatggaaaacaacagcggctggaaagtgagcctgaactatggcgaaattatttggaccctgcaagatacccaagaaattaaacagcgcgtggtgtttaaatatagtcagatgattaacattagcgattatattaaccgctggatttttgtgaccattaccaacaaccgtctgaacaacagcaaaatttatattaacggccgcctgattgatcagaaaccgattagcaacctgggcaacattcatgcgagcaacaacattatgtttaaactggatggcgagcgcgatacgcatcgctatatctggattaaatattttaatctgttcgacaaagaactgaacgaaaaagaaattaaagatctgtatgataatcagagcaacagcggcattctgaaagatttttggggcgattatctgcagtatgataaaccgtattatatgctgaacctgtatgatccgaacaaatatgtggatgtgaacaacgtgggcattcgcggctatatgtatctgaaaggcccgcgcggcagcgtgatgaccaccaacatttatctgaacagcagcctgtatcgcggcaccaaatttattattaaaaaatatgcgagcggcaacaaagataacattgtgcgcaacaacgatcgcgtgtatattaacgtggttgtgaaaaacaaagaatatcgcctggcgaccaacgcgagccaagcgggcgtggaaaaaattctgagcgcgctggaaattccggatgtgggcaacctgagccaagtggttgtgatgaaaagcaaaaacgatcaaggcattaccaacaagtgcaaaatgaacctgcaagataacaacggcaacgatattggctttattggctttcatcagtttaacaacattgcgaaactggtggcgagcaactggtataaccgtcagattgaacgcagcagccgcaccctgggctgcagctgggaatttattccggttgatgatggctggggcgaacgcccgctgtaa

SEQ ID NO: 11 - DNA, 1347 bp, synthetic construct
  source 1..1347: mol_type = other DNA
atgccgtttgtgaacaaacagtttaactataaagatccggtgaacggcgtggatattgcgtatattaaaattccgaacgcgggtcagatgcagccggtgaaagcgtttaaaattcataacaaaatttgggtgattccggaacgcgatacctttaccaacccggaagaaggcgatctgaacccgccaccggaagcgaaacaagtgccggtgagctattatgatagcacctatctgagcaccgataacgaaaaagataactatctgaaaggcgtgaccaaactgtttgaacgcatttatagcaccgatctgggccgcatgctgctgacgagcattgtgcgcggcattccgttctggggcggcagcaccattgataccgaactgaaagtgattgataccaacggtattaacgtgattcagccggatggcagctatcgcagcgaagaactgaacctggtgattattggcccgagcgcggatattattcagtttgaaggtaaaagctttggccatgaagtgctgaacctgacccgcaacggctatggcagcacgcagtatattcgctttagcccggattttacctttggctttgaagaaagcctggaagtggataccaacccgctgctgggcgcgggcaaatttgcgaccgatccggcggtgaccctggcgcatgaactgattcatgcgggccatcgcctgtatggcattgcgattaacccgaaccgcgtgtttaaagtgaacaccaacgcgtattatgaaatgagcggcctggaagtgagctttgaagaactgcgcacctttggcggccatgatgcgaaatttattgatagcctgcaagaaaacgaatttcgcctgtattactataacaaatttaaagatattgcgagcaccctgaacaaagcgaaaagcattgtgggcaccaccgcgagcctgcagtatatgaaaaacgtgtttaaagaaaaatatctgctgagcgaagatacgagcggcaaatttagcgtggataaactgaaatttgataaactgtataaaatgctgaccgaaatttataccgaagataactttgtgaaattttttaaagtgctgaaccgcaagacctatctgaactttgataaagcggtgtttaaaattaacattgtgccgaaagtgaactataccatttatgatggctttaacctgcgcaacaccaacctggcggcgaactttaacggtcagaacaccgaaattaacaacatgaactttaccaaactgaaaaactttaccggcctgtttgaattttataaactgctgtgcgttcgcggcatcattacgagcaaaaccaaaagcctggataaaggctataacaaataa

SEQ ID NO: 12 - DNA, 2550 bp, synthetic construct
  source 1..2550: mol_type = other DNA
atggcgctgaacgatctgtgcattaaagtgaataattgggatctgttttttagcccgagcgaagataactttaccaacgatctgaacaaaggcgaagaaattacgagcgataccaacattgaagcggcggaagagaacattagtctggatctgattcagcagtattatctgacctttaactttgataacgaaccggaaaacattagtattgaaaacctgagcagcgatattattggtcagctggaactgatgccgaacattgaacgctttccgaacggcaaaaaatatgaactggataaatataccatgtttcattatctgcgcgcgcaagaatttgaacatggcaaaagccgcattgcgctgaccaacagcgtgaacgaagcgctgctgaacccgagccgcgtgtataccttttttagcagcgattatgtgaaaaaagtgaacaaagcgaccgaagcggcgatgtttctgggctgggtggaacagctggtgtatgattttaccgatgagacgagcgaagtgagtaccaccgataaaattgcggatattaccattatcattccgtatattggcccggcgctgaacattggcaacatgctgtataaagatgattttgtgggcgcgctgatttttagcggcgcggtgattctgctggaatttattccggaaatcgcgattccggtgctgggcacctttgcgctggtgagctatattgcgaacaaagtgctgaccgtgcagaccattgataacgcgctgagcaaacgcaacgaaaaatgggatgaagtgtataaatatattgtgaccaactggctggcgaaagtgaacacgcagattgatctgattcgcaaaaaaatgaaagaagcgctggaaaaccaagcggaagcgaccaaggcgattattaactatcagtataatcagtataccgaagaggaaaaaaacaacattaactttaacattgatgatctgagcagcaaattaaatgaaagcattaacaaagcgatgatcaacattaacaagtttctgaatcaggcaagcgtgagctatctgatgaacagcatgattccgtatggcgtgaaacgcctggaagattttgatgcgagcctgaaagatgcgctgctgaaatatatttatgataaccgcggcaccctgattggccaagtggatcgcctgaaagataaagttaataacacgctgagcaccgatattccgtttcagctgagcaaatatgtggataatcagcgcctgctgagcacctttaccgaatatattaaaaacattattaacacgagcattctgaacctgcgctatgaaagcaaccatctgattgatctgagccgctatgcgagcaaaattaacattggcagcaaagtgaactttgatccgattgataaaaatcagattcagctgtttaacctggaaagcagcaaaattgaagtgattctgaaaaacgcgattgtgtataacagcatgtatgaaaactttagcacgagcttttggattcgcattccgaaatactttaacagcatcagcctgaacaacgaatataccattattaacgcaatggaaaacaacagcggctggaaagtgagcctgaactatggcgaaattatttggaccctgcaagatacccaagaaattaaacagcgcgtggtgtttaaatatagtcagatgattaacattagcgattatattaaccgctggatttttgtgaccattaccaacaaccgtctgaacaacagcaaaatttatattaacggccgcctgattgatcagaaaccgattagcaacctgggcaacattcatgcgagcaacaacattatgtttaaactggatggcggtcgcgatacgcatcgctatatctggattaaatattttaatctgttcgacaaagaactgaacgaaaaagaaattaaagatctgtatgataatcagagcaacagcggcattctgaaagatttttggggcgattatctgcagtatgataaaccgtattatatgctgaacctgtatgatccgaacaaatatgtggatgtgaacaacgtgggcattcgcggctatatgtatctgaaaggcccgcgcggcagcgtgatgaccaccaacatttatctgaacagcagcctgtatcgcggcaccaaatttattattaaaaaatatgcgagcggcaacaaagataacattgtgcgcaacaacgatcgcgtgtatattaacgtggttgtgaaaaacaaagaatatcgcctggcgaccaacgcgagccaagcgggcgtggaaaaaattctgagcgcgctggaaattccggatgtgggcaacctgagccaagtggttgtgatgaaaagcaaaaacgatcaaggcattaccaacaagtgcaaaatgaacctgcaagataacaacggcaacgatattggctttattggctttcatcagtttaacaacattgcgaaactggtggcgagcaactggtataaccgtcagattgaacgcagcagccgcaccctgggctgcagctgggaatttattccggttgatgatggctggggcgaacgcccgctgtaa

(19) United States
(12) Patent Application Publication
(10) Pub. No.: US 2024/0240168 A1
(43) Pub. Date: Jul. 18, 2024

(54) RECOMBINANT BOTULINUM NEUROTOXIN OF TYPE A AND PREPARATION METHOD THEREOF

(71) Applicant: JHM BIOPHARMACEUTICAL (HANGZHOU) CO., LTD., Hangzhou (CN)

(72) Inventors: Yubao SHEN, Hangzhou (CN); Guoqing BAO, Hangzhou (CN); Liming GUO, Hangzhou (CN)

(73) Assignee: JHM BIOPHARMACEUTICAL (HANGZHOU) CO., LTD., Hangzhou (CN)

(21) Appl. No.: 18/614,059

(22) Filed: Mar. 22, 2024

Related U.S. Application Data
(63) Continuation of application No. PCT/CN2023/095060, filed on May 18, 2023.

(30) Foreign Application Priority Data
May 24, 2022 (CN) 202210572145.3
Sep. 13, 2022 (CN) 202211112222.3

(51) Int. Cl. C12N 9/52; A61K 38/00

(52) U.S. Cl. CPC C12N 9/52; C12Y 304/24069; A61K 38/00

(57) ABSTRACT

The present disclosure relates to a recombinant BoNT/A and a preparation method thereof. The recombinant BoNT/A is a BoNT/A mutant, the BoNT/A mutant comprising a first peptide fragment and a second peptide fragment, which are linked through an interchain disulfide bond; wherein the first peptide fragment has a mutation at position 134 and/or position 165 compared to the light chain of a wild-type BoNT/A; and/or the second peptide fragment has at least one of the following mutation positions compared to the heavy chain of the wild-type BoNT/A: positions 791, 967, and 1060. The method includes: subjecting the first peptide fragment to a first renaturation treatment to obtain a first denatured product, subjecting the second peptide fragment to a second denaturation treatment to obtain a second denatured product, and subjecting the first denatured product and the second denatured product to a renaturation and assembly treatment to obtain the BoNT/A mutant.

### CROSS-REFERENCE TO RELATED APPLICATIONS

This application is a continuation of International Application No. PCT/CN2023/095060, filed on May 18, 2023, which claims priority to Chinese Patent Application No. 202210572145.3, filed on May 24, 2022 and Chinese Patent Application No. 202211112222.3, filed on Sep. 13, 2022. The disclosures of the aforementioned applications are hereby incorporated by reference in their entireties.

### STATEMENT REGARDING SEQUENCE LISTING

A Sequence Listing associated with this application is being filed concurrently herewith in XML format and is hereby incorporated by reference into the present specification. The XML file containing the Sequence listing is titled “Sequence_Listing.xml”, was created on Jan. 31, 2024, and is approximately 27 kilobytes in size.

### FIELD

The present application relates to the field of biopharmaceutical technology. In particular, the present disclosure relates to a recombinant botulinum neurotoxin (BoNT) and a preparation method thereof. In particular, the present disclosure relates to a BoNT/A, a mutant thereof, and a preparation method thereof.

### BACKGROUND

Botulinum neurotoxin (BoNT), as a neurotoxin produced by anaerobic Clostridium botulimim (referred to as “C. botulimim”), is one of the most toxic substances known in the world. The BoNTs can be divided into seven serotypes, i.e., type A to type G, the most common of which is type A, called Botulinum neurotoxin of type A (BoNT/A).

The BoNT/A can be structurally divided into two parts: a light chain (LC, 50 kD) and a heavy chain (HC, 100 kD), which are linked by a pair of interchain disulfide bond (C430-C454), a non-amide bond. The light chain is an active domain with zinc-dependent metallopeptidase activity and is a toxic part of the botulinum toxin. The heavy chain includes two domains, i.e., a binding domain responsible for binding to a corresponding receptor on the membrane of the nerve cell and forming an ion channel on the endosomal membrane, and a translocation domain responsible for translocating the light chain into the cell. The light chain recognizes and specifically cleaves the Q197-R198 position (a synaptic vesicle-associated protein) on SNAP-25.

A number of studies indicate that BoNT/A has many application scenarios, not only being applied in the medical cosmetology, but also having broad application prospects in the treatment of various diseases. With the clinical application in various areas such as muscle tone disorders, hand and foot hyperhidrosis, pains, and various other difficult and complex diseases, the range of application in the disease treatment is constantly expanding. The therapeutic mechanism of BoNT/A is in that BoNT/A binds to a corresponding receptor on the surface of the nerve cells at the neuromuscular junction. By means of its heavy chain N-terminal, the light chain is transferred across the membrane into the cells. The release of acetylcholine is blocked by cleaving SNAP-25, causing sustained muscle relaxation paralysis.

A naturally structured active BoNT/A protein has a pair of disulfide bonds (C1235-C1280) within the heavy chain and a pair of disulfide bonds (C430-C454) between the light chain and heavy chain. In addition to four cysteines forming intrachain and interchain disulfide bonds, the molecular structure of BoNT/A further contain other five free cysteines (non-pairing to form a disulfide bond, the same as below). During the preparation of recombinant BoNT/A, the mismatch of disulfide bonds is prone to occur, resulting in the formation of BoNT/A protein with incorrect structure, leading to a decrease in the activity of BoNT/A or even resulting in an immune response when applied to the human body.

Therefore, there is an urgent need to develop a BoNT/A with correct spatial structure and high biological activity.

### SUMMARY

The present disclosure aims to solve at least one of the technical problems in the related technology to a certain extent. To this end, the present disclosure provides a BoNT/A mutant having the advantages of low mismatch rate of disulfide bonds and high biological activity (virulence).

The present disclosure was accomplished based on the inventors' following findings.

The naturally structured active BoNT/A is composed of 1296 amino acids, and is divided into a light chain and a heavy chain. The light chain contains 448 amino acids (1-448), and the heavy chain contains 848 amino acids (449-1296). A pair of disulfide bonds (C1235-C1280) is formed in the heavy chain and located at a proximal C-terminal region of the amino acid sequence of the heavy chain. A pair of disulfide bonds (C430-C454) is formed between the light chain and the heavy chain, and cysteines forming the disulfide bonds are respectively located at a proximal C-terminal of an amino acid sequence of the light chain and a proximal N-terminal of the amino acid sequence of the heavy chain. By virtue of the disulfide bonds, the light and heavy chains form the correct architecture of the whole BoNT/A molecule, enabling the BoNT/A molecule to having biological effects such as binding to receptors, trans-membrane transport and playing virulence.

In addition to four cysteines forming intrachain and interchain disulfide bonds (C1235-C1280) and the disulfide bonds between the light and heavy chains (C430-C454), a primary structure of the light and heavy chains further contain 5 free cysteines in an unpaired state. The production of BoNT/A by Clostridium botulinum in nature is a slow process. The molecular force of botulinum toxin for forming the natural structure tends to cause BoNT/A in nature to form the correct higher-order conformation. In the process of artificially culturing Clostridium botulinum or preparing BoNT/A by using other expression modes (such as using Escherichia coli expression), since the natural growth law of Clostridium botulinum or the formation rate of BoNT/A in other host bacteria is artificial intervened, some BoNT/A in the host cells cannot form the correct high-order conformation consistent with that in the natural state, thereby affecting the virulence of BoNT/A. In addition to the effects of intermolecular forces (such as hydrogen bond, van der Waals force, hydrophobic interaction, electrostatic interaction, etc.), the spatial configuration of BoNT/A may be also changed to some extent due to an erroneous disulfide bond caused by the spatial mismatch between free cysteines, resulting in that a C-terminal receptor binding region of the heavy chain, or a N-terminal translocation region of the heavy chain, or an active region of the light chain of the intact BoNT/A molecule no longer has the high-order spatial configuration of the subunit structure of the natural BoNT/A molecule, thereby reducing or weakening the biological effect of the BoNT/A molecule in vivo. Due to changes in terms of the high-order structure or spatial conformation of intact molecules or subunits, the human body may even produce anti-protein antibodies (APA) against these unnatural high-order structures or spatial conformations after administration.

The formation of forces in the molecular structure is closely related to the transcription rate, translation efficiency, and redox environment in the host cells during the formation of the BoNT/A molecule. Excessively high or unmatched transcription rate, translation efficiency, and unsuitable host cell environment may likely lead to the incorrect structure of BoNT/A molecule. In the process of in vivo renaturation and assembly, the presence of free cysteines may likely lead to mismatch of disulfide bond between cysteine molecules, which may occur either between free cysteines at natural structural positions, or between free cysteines at natural structural positions and cysteines with pairing positions at natural structural positions, or between cysteines with different pairing positions at natural structural positions. In any case, it will change the high-order conformation of BoNT/A molecules to some extent, thereby affecting the biological effects of BoNT/A molecules in vivo. Thus, the production of APA against these “unnatural” structures may be increased after the administration to human body, reducing efficacy.

Accordingly, in an aspect of the present disclosure, a BoNT/A mutant is provided. According to an embodiment of the present disclosure, the BoNT/A mutant includes a first peptide fragment and a second peptide fragment. The first peptide fragment and the second peptide fragment are linked through an interchain disulfide bond. The first peptide fragment has a mutation at position 134 and/or position 165 compared to the light chain of a wild-type BoNT/A; and/or the second peptide fragment has at least one of the following mutation positions compared to the heavy chain of the wild-type BoNT/A: positions 791, 967, and 1060. The inventors have analyzed the molecular structure of native BoNT/A and found through extensive experiments that mutation of the above-mentioned amino acids of BoNT/A can reduce disulfide bond mismatch and increase the biological activity thereof.

In another aspect of the present disclosure, a nucleic acid molecule is provided. According to an embodiment of the present disclosure, the nucleic acid molecule encodes the first and/or the second peptide fragment of the aforementioned BoNT/A mutant. The nucleic acid molecules according to an embodiment of the present disclosure may encode the aforementioned BoNT/A mutant.

In another aspect of the present disclosure, an expression vector is provided. According to an embodiment of the present disclosure, the expression vector carries the aforementioned nucleic acid molecule. The inventors have found through experiments that the expression of the BoNT/A mutant can be efficiently achieved under the mediation of a regulatory system by transforming the aforementioned expression vector into suitable host bacteria, thereby obtaining the BoNT/A mutant in large quantities.

In another aspect of the present disclosure, genetic engineering bacteria are provided. According to an embodiment of the present disclosure, the genetic engineering bacteria include: genetic engineering bacteria carrying the aforementioned nucleic acid molecule or the aforementioned expression vector; or genetic engineering bacteria expressing the aforementioned BoNT/A mutant. The inventors have found through experiments that the aforementioned BoNT/A mutant can be efficiently expressed when the above-mentioned genetic engineering bacteria are cultured under appropriate conditions.

In another aspect of the present disclosure, a method for preparing BoNT/A by gene recombination is provided. According to an embodiment of the present disclosure, the method includes: performing a first denaturation treatment on a light chain protein to obtain a first denatured product; performing a second denaturation treatment on a heavy chain protein to obtain a second denatured product; and performing a renaturation and assembly treatment on the first denatured product and the second denatured product to obtain the BoNT/A.

The preparation method according to the present disclosure can prepare a single subtype and single component BoNT/A protein free of sequence heterogeneity and having a correct conformation. The BoNT/A protein is free of HA, NTNH, which may be contained in the Clostridium botulinum extract, and is also free of non-BoNT/A components such as enzyme residues, which may be produced when activating the toxicity of botulinum toxin using protease subsequent to the expression of the intact BoNT/A protein. The method has the advantage of stable batch-to-batch quality and is more suitable for commercial-scale production. In addition, the preparation method is simple, high-efficient, and short in the preparation process, and the whole preparation process can be finished in only 8 steps within 4 to 5 days.

In another aspect of the present disclosure, a pharmaceutical composition is provided. According to an embodiment of the present disclosure, the pharmaceutical composition includes the aforementioned BoNT/A mutant. The pharmaceutical composition according to the present disclosure has high biological activity (virulence). It can be used not only for medical cosmetology but also for the treatment or improvement of dystonia, hyperhidrosis, or pains, etc.

In another aspect of the present disclosure, provided are uses of the aforementioned BoNT/A mutant, BoNT/A prepared by the aforementioned method, or the aforementioned pharmaceutical composition in the preparation of a medicament. The medicament can be used in medical cosmetology; or the medicament can used in the treatment or amelioration of at least one of strabismus, cervical dystonia, laryngeal dystonia, upper limb focal dystonia, primary hand tremor, sialorrhea, blepharospasm, hemifacial spasm, stroke-caused upper/lower limb spasm, cerebral palsy-caused upper/lower limb spasm, axillary hyperhidrosis, palmar hyperhidrosis, detrusor-sphincter dyssynergia, chronic migraine, and neurogenic and idiopathic overactive bladder.

In another aspect of the present disclosure, provided are uses of the aforementioned BoNT/A mutant, BoNT/A prepared by the aforementioned method, or the aforementioned pharmaceutical composition in medical cosmetology or the treatment or amelioration of a disease. The disease includes at least one of strabismus, cervical dystonia, laryngeal dystonia, upper limb focal dystonia, primary hand tremor, sialorrhea, blepharospasm, hemifacial spasm, stroke-caused upper/lower limb spasm, cerebral palsy-caused upper/lower limb spasm, axillary hyperhidrosis, palmar hyperhidrosis, detrusor-sphincter dyssynergia, chronic migraine, and neurogenic and idiopathic overactive bladder.

In another aspect of the present disclosure, provided is the aforementioned BoNT/A mutant, BoNT/A prepared by the aforementioned method, or the aforementioned pharmaceutical composition in medical cosmetology for use in the treatment or amelioration of a disease. The disease includes at least one of strabismus, cervical dystonia, laryngeal dystonia, upper limb focal dystonia, primary hand tremor, sialorrhea, blepharospasm, hemifacial spasm, stroke-caused upper/lower limb spasm, cerebral palsy-caused upper/lower limb spasm, axillary hyperhidrosis, palmar hyperhidrosis, detrusor-sphincter dyssynergia, chronic migraine, and neurogenic and idiopathic overactive bladder.

In another aspect of the present disclosure, the present disclosure provides a method for ameliorating and/or treating a disease. According to an embodiment of the present disclosure, the method includes: administrating to a subject the aforementioned BoNT/A mutant, BoNT/A prepared by the aforementioned method, or the aforementioned pharmaceutical composition. The disease includes at least one of strabismus, cervical dystonia, laryngeal dystonia, upper limb focal dystonia, primary hand tremor, sialorrhea, blepharospasm, hemifacial spasm, stroke-caused upper/lower limb spasm, cerebral palsy-caused upper/lower limb spasm, axillary hyperhidrosis, palmar hyperhidrosis, detrusor-sphincter dyssynergia, chronic migraine, and neurogenic and idiopathic overactive bladder.

In another aspect of the present disclosure, a medical cosmetology is provided. According to an embodiment of the present disclosure, the method includes: administrating to a subject the aforementioned BoNT/A mutant, BoNT/A prepared by the aforementioned method, or the aforementioned pharmaceutical composition.

### BRIEF DESCRIPTION OF DRAWINGS

FIG. 1 is an SDS-PAGE image of BoNT/A-LC expression, BoNT/A-HC expression, and inclusion bodies in Example 2 of the present disclosure;

FIG. 2 is an SDS-PAGE image of a sample after renaturation and assembly in Example 2 of the present disclosure;

FIG. 3 is an SDS-PAGE image of BoNT/A protein in Example 2 of the present disclosure;

FIG. 4 illustrates an SEC chromatographic purity of BoNT/A protein in Example 2 of the present disclosure;

FIG. 5 illustrates complete molecular weight data analysis diagrams in a structural identification of Example 3 of the present disclosure;

FIG. 6 illustrates reduced molecular weight data analysis diagrams in a structural identification of Example 3 of the present disclosure;

FIG. 7 illustrates an analysis diagram of disulfide bond positions in a structural identification of Example 3 of the present disclosure;

FIG. 8 illustrates a double digestion agarose electrophoresis of a light chain mutant plasmid and a double digestion agarose electrophoresis of a heavy chain mutant plasmid according to Example 6 of the present disclosure; and

FIG. 9 illustrates positions of cysteines and link position of disulfide bonds in the wild-type BoNT/A in Example 7 of the present disclosure.

### DETAILED DESCRIPTION OF EMBODIMENTS

The embodiments of the present disclosure are described in detail below. The embodiments described below are merely illustrative, and they cannot be construed as limiting the present disclosure. Where specific techniques or conditions are not specified in the examples, they are performed according to techniques or conditions described in the literature in the related art or according to the product specification. The used reagents or instruments without specifying the manufacturer are all conventional and commercially available products.

It should be noted that the terms “first” and “second” are used for descriptive purposes only and shall not to be construed as indicating or implying relative importance or implicitly indicating the number of technical features indicated. Thus, a feature defined as “first” or “second” may explicitly or implicitly include one or more of the features. Further, in the description of the present disclosure, unless otherwise specified, the meaning of “a plurality” is two or more.

The endpoints of the ranges and any values disclosed herein are not limited to the precise range or value, and these ranges or values should be understood to include values approximating such ranges or values. For numerical ranges, the endpoints of each range, the endpoints of each range and the individual point values, and the individual point values, can be combined with each other to produce one or more new numerical ranges, and such numerical ranges are to be considered to be specifically disclosed herein.

For an easier understanding of the present disclosure, certain technical and scientific terms are specifically defined below. Unless clearly defined elsewhere in this document, all other technical and scientific terms used in this document have the meanings commonly understood by general technical personnel in the field to which the present disclosure belongs.

In this document, the terms “comprising” or “including” are used in an open-ended expression, i.e., including what is specified in the present disclosure, but not excluding other aspects.

As used herein, the terms “optionally”, “optional” or “option” generally mean that the subsequent events or conditions may but may not necessarily occur, and the description includes the circumstances in which the event or condition occurred, as well as the circumstances in which the event or condition did not occur.

In this context, the amino acid numbering of the BoNT/A is according to the EU numbering system. For example, position 134 refers to the 134-th position according to the EU numbering system; the “C134G” refers to that cysteine at position 134 according to the EU numbering system is substituted with glycine; and “C791A” refers to that cysteine at position 791 according to the EU numbering system is substituted with alanine.

As used herein, the term “expression vector” generally refers to a vector of nucleic acid molecule, which is inserted or insertable in a suitable host for self-replicating, i.e., transferring the inserted nucleic acid molecule into a host organism. The expression vector may include a vector mainly for inserting DNA or RNA into cells, a vector mainly for replicating DNA or RNA, and a vector mainly for expressing transcription and/or translation of DNA or RNA, preferably DNA. The expression vectors may further include vectors having many of the functions as described above, and types of expression vectors include, but are not limited to, plasmids, linear DNA fragments, viruses, bacteriophages, proviruses, phagemids, transposons, artificial chromosomes, and the like. The expression vector contains target gene fragment, which can be transcribed and translated into amino acids that make up polypeptide when the expression vector being transformed into a suitable host organism. Typically, the expression vector may produce the desired expression product by culturing suitable host bacteria containing the expression vector.

As used herein, the term “genetic engineering bacteria” generally refers to bacteria that transform expression vector containing target gene into the host bacteria to express the target gene and produce the desired protein. Herein, the term “host bacteria” refer to a bacteria or cells, such as Escherichia coli (E. coli), into which a recombinant expression vector can be transformed.

As used herein, the term “pharmaceutical composition” generally refers to a unit dosage form and may be prepared by any of the methods well known in the pharmaceutical art. All methods include a step of combining an active ingredient with a vehicle that constitutes one or more vehicle ingredients. In general, the composition is prepared by uniformly and intimately bringing the active compound in combination with a liquid vehicle, finely divided solid vehicle, or both.

As used herein, the term “pharmaceutically acceptable adjuvant” may include any solvent, solid excipient, diluent, or other liquid excipient, etc., which is suitable for the particular desired dosage form. Except the conventional adjuvant incompatible with the compounds of the present disclosure, for example, by producing any undesirable biological effect or interacting in a deleterious manner with any other component (s) of the pharmaceutically acceptable composition, use thereof is contemplated to be within the scope of the present disclosure.

As used herein, the term “treatment” refers to obtaining a desired pharmacologic and/or physiologic effect. The effect may be prophylactic in terms of completely or partially preventing or ameliorating a disease or symptom thereof and/or may be therapeutic in terms of a partial or complete cure for a disease and/or adverse effect attributable to the disease. As used herein, the term “treatment” encompasses diseases of mammals, particularly human being, including (a) preventing the disease or condition from occurring in an individual who is predisposed to the disease but has not yet been diagnosed with the disease; (b) inhibiting the disease, for example, arresting the development of the disease; or (c) relieving the disease, e.g., alleviating symptoms associated with the disease. As used herein, the term “treatment” encompasses treating, curing, alleviating, ameliorating, lessening, or inhibiting disease in a subject by administrating a drug or compound to the subject, including, but not limited to, administrating the drug including the compound described herein to the subject in need thereof.

As used herein, the terms “BoNT/A”, “BoNT/A protein”, “BoNT/A component”, “BoNT/A molecule” and “botulinum neurotoxin of type A protein” all refer to Botulinum neurotoxin of type A.

As used herein, the terms “LC”, “BoNT/A-LC”, “BoNT/A-LC protein”, “light chain protein”, and “light chain” are synonymous. The terms “HC”, “BoNT/A-HC”, “BoNT/A-HC protein”, “heavy chain protein” and “heavy chain” are synonymous. The terms “light chain protein” and “light chain” are synonymous. The terms “heavy chain protein” and “heavy chain” are synonymous. The terms “light chain mutant protein” and “light chain mutant” are synonymous, and the terms “heavy chain mutant protein” and “heavy chain mutant” are synonymous.

The present disclosure provides a BoNT/A mutant, a nucleic acid molecule, an expression vector, genetic engineering bacteria, a method for producing a BoNT/A by gene recombination, a pharmaceutical composition, and uses thereof, each of which is described in detail below.

### BoNT/A Mutant

In a first aspect, a BoNT/A mutant is provided. According to an embodiment of the present disclosure, the BoNT/A mutant includes a first peptide fragment and a second peptide fragment. The first peptide fragment and the second peptide fragment are linked through an interchain disulfide bond. The first peptide fragment has a mutation at position 134 and/or position 165 compared to a light chain of a wild-type BoNT/A; and/or the second peptide fragment has at least one of the following mutation positions compared to a heavy chain of the wild-type BoNT/A: positions 791, 967, and 1060. The inventors have analyzed the structure of the native BoNT/A molecule and have found through extensive experiments that mutation of the above amino acids of BoNT/A reduces disulfide bond mismatch and such BoNT/A mutants have high biological activity (virulence).

The light chain of the wild-type BoNT/A has an amino acid sequence as set forth in SEQ ID NO: 1:

(SEQ ID NO: 1)
MPFVNKQFNYKDPVNGVDIAYIKIPNAGQMQPVKAFKIHNKIWVI
PERDTFTNPEEGDLNPPPEAKQVPVSYYDSTYLSTDNEKDNYLKG
VTKLFERIYSTDLGRMLLTSIVRGIPFWGGSTIDTELKVIDTNCI
NVIQPDGSYRSEELNLVIIGPSADIIQFECKSFGHEVLNLTRNGY
GSTQYIRFSPDFTFGFEESLEVDTNPLLGAGKFATDPAVTLAHEL
IHAGHRLYGIAINPNRVFKVNTNAYYEMSGLEVSFEELRTFGGHD
AKFIDSLQENEFRLYYYNKFKDIASTLNKAKSIVGTTASLQYMKN
VFKEKYLLSEDTSGKFSVDKLKFDKLYKMLTEIYTEDNFVKFFKV
LNRKTYLNFDKAVFKINIVPKVNYTIYDGFNLRNTNLAANFNGQN
TEINNMNFTKLKNFTGLFEFYKLLCVRGIITSKTKSLDKGYNK;

The heavy chain of the wild-type BoNT/A has an amino acid sequence as set forth in SEQ ID NO: 2:

(SEQ ID NO: 2)
ALNDLCIKVNNWDLFFSPSEDNFTNDLNKGEEITSDTNIEAAEEN
ISLDLIQQYYLTFNFDNEPENISIENLSSDIIGQLELMPNIERFP
NGKKYELDKYTMFHYLRAQEFEHGKSRIALTNSVNEALLNPSRVY
TFFSSDYVKKVNKATEAAMFLGWVEQLVYDFTDETSEVSTTDKIA
DITIIIPYIGPALNIGNMLYKDDFVGALIFSGAVILLEFIPEIAI
PVLGTFALVSYIANKVLTVQTIDNALSKRNEKWDEVYKYIVTNWL
AKVNTQIDLIRKKMKEALENQAEATKAIINYQYNQYTEEEKNNIN
FNIDDLSSKLNESINKAMININKFLNQCSVSYLMNSMIPYGVKRL
EDFDASLKDALLKYIYDNRGTLIGQVDRLKDKVNNTLSTDIPFQL
SKYVDNQRLLSTFTEYIKNIINTSILNLRYESNHLIDLSRYASKI
NIGSKVNFDPIDKNQIQLFNLESSKIEVILKNAIVYNSMYENFST
SFWIRIPKYFNSISLNNEYTIINCMENNSGWKVSLNYGEIIWTLQ
DTQEIKQRVVFKYSQMINISDYINRWIFVTITNNRLNNSKIYING
RLIDQKPISNLGNIHASNNIMFKLDGCRDTHRYIWIKYFNLFDKE
LNEKEIKDLYDNQSNSGILKDFWGDYLQYDKPYYMLNLYDPNKYV
DVNNVGIRGYMYLKGPRGSVMTTNIYLNSSLYRGTKFIIKKYASG
NKDNIVRNNDRVYINVVVKNKEYRLATNASQAGVEKILSALEIPD
VGNLSQVVVMKSKNDQGITNKCKMNLQDNNGNDIGFIGFHQFNNI
AKLVASNWYNRQIERSSRTLGCSWEFIPVDDGWGERPL.

The nucleotide sequence encoding the wild-type BoNT/A light chain is set forth in SEQ ID NO: 7:

(SEQ ID NO: 7)
ATGCCGTTTGTGAACAAACAGTTTAACTATAAAGATCCGGTGAAC
GGCGTGGATATTGCGTATATTAAAATTCCGAACGCGGGTCAGATG
CAGCCGGTGAAAGCGTTTAAAATTCATAACAAAATTTGGGTGATT
CCGGAACGCGATACCTTTACCAACCCGGAAGAAGGCGATCTGAAC
CCGCCACCGGAAGCGAAACAAGTGCCGGTGAGCTATTATGATAGC
ACCTATCTGAGCACCGATAACGAAAAAGATAACTATCTGAAAGGC
GTGACCAAACTGTTTGAACGCATTTATAGCACCGATCTGGGCCGC
ATGCTGCTGACGAGCATTGTGCGCGGCATTCCGTTCTGGGGCGGC
AGCACCATTGATACCGAACTGAAAGTGATTGATACCAACTGCATT
AACGTGATTCAGCCGGATGGCAGCTATCGCAGCGAAGAACTGAAC
CTGGTGATTATTGGCCCGAGCGCGGATATTATTCAGTTTGAATGC
AAAAGCTTTGGCCATGAAGTGCTGAACCTGACCCGCAACGGCTAT
GGCAGCACGCAGTATATTCGCTTTAGCCCGGATTTTACCTTTGGC
TTTGAAGAAAGCCTGGAAGTGGATACCAACCCGCTGCTGGGCGCG
GGCAAATTTGCGACCGATCCGGCGGTGACCCTGGCGCATGAACTG
ATTCATGCGGGCCATCGCCTGTATGGCATTGCGATTAACCCGAAC
CGCGTGTTTAAAGTGAACACCAACGCGTATTATGAAATGAGCGGC
CTGGAAGTGAGCTTTGAAGAACTGCGCACCTTTGGCGGCCATGAT
GCGAAATTTATTGATAGCCTGCAAGAAAACGAATTTCGCCTGTAT
TACTATAACAAATTTAAAGATATTGCGAGCACCCTGAACAAAGCG
AAAAGCATTGTGGGCACCACCGCGAGCCTGCAGTATATGAAAAAC
GTGTTTAAAGAAAAATATCTGCTGAGCGAAGATACGAGCGGCAAA
TTTAGCGTGGATAAACTGAAATTTGATAAACTGTATAAAATGCTG
ACCGAAATTTATACCGAAGATAACTTTGTGAAATTTTTTAAAGTG
CTGAACCGCAAGACCTATCTGAACTTTGATAAAGCGGTGTTTAAA
ATTAACATTGTGCCGAAAGTGAACTATACCATTTATGATGGCTTT
AACCTGCGCAACACCAACCTGGCGGCGAACTTTAACGGTCAGAAC
ACCGAAATTAACAACATGAACTTTACCAAACTGAAAAACTTTACC
GGCCTGTTTGAATTTTATAAACTGCTGTGCGTTCGCGGCATCATT
ACGAGCAAAACCAAAAGCCTGGATAAAGGCTATAACAAATAA

The nucleotide sequence encoding the wild-type BoNT/A heavy chain is set forth in SEQ ID NO: 8:

(SEQ ID NO: 8)
ATGGCGCTGAACGATCTGTGCATTAAAGTGAATAATTGGGATCTG
TTTTTTAGCCCGAGCGAAGATAACTTTACCAACGATCTGAACAAA
GGCGAAGAAATTACGAGCGATACCAACATTGAAGCGGCGGAAGAG
AACATTAGTCTGGATCTGATTCAGCAGTATTATCTGACCTTTAAC
TTTGATAACGAACCGGAAAACATTAGTATTGAAAACCTGAGCAGC
GATATTATTGGTCAGCTGGAACTGATGCCGAACATTGAACGCTTT
CCGAACGGCAAAAAATATGAACTGGATAAATATACCATGTTTCAT
TATCTGCGCGCGCAAGAATTTGAACATGGCAAAAGCCGCATTGCG
CTGACCAACAGCGTGAACGAAGCGCTGCTGAACCCGAGCCGCGTG
TATACCTTTTTTAGCAGCGATTATGTGAAAAAAGTGAACAAAGCG
ACCGAAGCGGCGATGTTTCTGGGCTGGGTGGAACAGCTGGTGTAT
GATTTTACCGATGAGACGAGCGAAGTGAGTACCACCGATAAAATT
GCGGATATTACCATTATCATTCCGTATATTGGCCCGGCGCTGAAC
ATTGGCAACATGCTGTATAAAGATGATTTTGTGGGCGCGCTGATT
TTTAGCGGCGCGGTGATTCTGCTGGAATTTATTCCGGAAATCGCG
ATTCCGGTGCTGGGCACCTTTGCGCTGGTGAGCTATATTGCGAAC
AAAGTGCTGACCGTGCAGACCATTGATAACGCGCTGAGCAAACGC
AACGAAAAATGGGATGAAGTGTATAAATATATTGTGACCAACTGG
CTGGCGAAAGTGAACACGCAGATTGATCTGATTCGCAAAAAAATG
AAAGAAGCGCTGGAAAACCAAGCGGAAGCGACCAAGGCGATTATT
AACTATCAGTATAATCAGTATACCGAAGAGGAAAAAAACAACATT
AACTTTAACATTGATGATCTGAGCAGCAAATTAAATGAAAGCATT
AACAAAGCGATGATCAACATTAACAAGTTTCTGAATCAGTGCAGC
GTGAGCTATCTGATGAACAGCATGATTCCGTATGGCGTGAAACGC
CTGGAAGATTTTGATGCGAGCCTGAAAGATGCGCTGCTGAAATAT
ATTTATGATAACCGCGGCACCCTGATTGGCCAAGTGGATCGCCTG
AAAGATAAAGTTAATAACACGCTGAGCACCGATATTCCGTTTCAG
CTGAGCAAATATGTGGATAATCAGCGCCTGCTGAGCACCTTTACC
GAATATATTAAAAACATTATTAACACGAGCATTCTGAACCTGCGC
TATGAAAGCAACCATCTGATTGATCTGAGCCGCTATGCGAGCAAA
ATTAACATTGGCAGCAAAGTGAACTTTGATCCGATTGATAAAAAT
CAGATTCAGCTGTTTAACCTGGAAAGCAGCAAAATTGAAGTGATT
CTGAAAAACGCGATTGTGTATAACAGCATGTATGAAAACTTTAGC
ACGAGCTTTTGGATTCGCATTCCGAAATACTTTAACAGCATCAGC
CTGAACAACGAATATACCATTATTAACTGCATGGAAAACAACAGC
GGCTGGAAAGTGAGCCTGAACTATGGCGAAATTATTTGGACCCTG
CAAGATACCCAAGAAATTAAACAGCGCGTGGTGTTTAAATATAGT
CAGATGATTAACATTAGCGATTATATTAACCGCTGGATTTTTGTG
ACCATTACCAACAACCGTCTGAACAACAGCAAAATTTATATTAAC
GGCCGCCTGATTGATCAGAAACCGATTAGCAACCTGGGCAACATT
CATGCGAGCAACAACATTATGTTTAAACTGGATGGCTGCCGCGAT
ACGCATCGCTATATCTGGATTAAATATTTTAATCTGTTCGACAAA
GAACTGAACGAAAAAGAAATTAAAGATCTGTATGATAATCAGAGC
AACAGCGGCATTCTGAAAGATTTTTGGGGCGATTATCTGCAGTAT
GATAAACCGTATTATATGCTGAACCTGTATGATCCGAACAAATAT
GTGGATGTGAACAACGTGGGCATTCGCGGCTATATGTATCTGAAA
GGCCCGCGCGGCAGCGTGATGACCACCAACATTTATCTGAACAGC
AGCCTGTATCGCGGCACCAAATTTATTATTAAAAAATATGCGAGC
GGCAACAAAGATAACATTGTGCGCAACAACGATCGCGTGTATATT
AACGTGGTTGTGAAAAACAAAGAATATCGCCTGGCGACCAACGCG
AGCCAAGCGGGCGTGGAAAAAATTCTGAGCGCGCTGGAAATTCCG
GATGTGGGCAACCTGAGCCAAGTGGTTGTGATGAAAAGCAAAAAC
GATCAAGGCATTACCAACAAGTGCAAAATGAACCTGCAAGATAAC
AACGGCAACGATATTGGCTTTATTGGCTTTCATCAGTTTAACAAC
ATTGCGAAACTGGTGGCGAGCAACTGGTATAACCGTCAGATTGAA
CGCAGCAGCCGCACCCTGGGCTGCAGCTGGGAATTTATTCCGGTT
GATGATGGCTGGGGCGAACGCCCGCTGTAA

According to an embodiment of the present disclosure, the interchain disulfide bond is formed by a cysteine at position 430 in the first peptide fragment and a cysteine at position 454 in the second peptide fragment.

According to an embodiment of the present disclosure, the first peptide fragment has mutations at positions 134 and 165 compared to the light chain of the wild-type BoNT/A; and/or the second peptide fragment has mutations at the positions 791, 967 and 1060 compared to the heavy chain of the wild-type BoNT/A. Thus, the mismatch rate of disulfide bonds in BoNT/A mutant can be reduced and the biological activity can be increased.

According to an embodiment of the present disclosure, cysteine at position 134, 165, 791, 967 or 1060 is mutated to one of the following amino acids: G, A, S, E, and P. Thus, the mismatch rate of disulfide bonds in BoNT/A mutant can be further reduced and the biological activity can be increased.

According to an embodiment of the present disclosure, the C at position 134 in the first peptide fragment is mutated to G, A, or S.

In an alternative embodiment of the present disclosure, the C at position 134 in the first peptide fragment is mutated to G.

In an alternative embodiment of the present disclosure, the C at position 134 in the first peptide fragment is mutated to A.

In an alternative embodiment of the present disclosure, the C at position 134 in the first peptide fragment is mutated to S.

According to an embodiment of the present disclosure, the C at position 165 in the first peptide fragment is mutated to G, A, P, or S.

In an alternative embodiment of the present disclosure, the C at position 165 in the first peptide fragment is mutated to G.

In an alternative embodiment of the present disclosure, the C at position 165 in the first peptide fragment is mutated to A.

In an alternative embodiment of the present disclosure, the C at position 165 in the first peptide fragment is mutated to P.

In an alternative embodiment of the present disclosure, the C at position 165 in the first peptide fragment is mutated to S.

According to an embodiment of the present disclosure, the C at position 791 in the second peptide fragment is mutated to G, A, or S.

In an alternative embodiment of the present disclosure, the C at position 791 in the second peptide fragment is mutated to G.

In an alternative embodiment of the present disclosure, the C at position 791 in the second peptide fragment is mutated to A.

In an alternative embodiment of the present disclosure, the C at position 791 in the second peptide fragment is mutated to S.

According to an embodiment of the present disclosure, the C at position 967 in the second peptide fragment is mutated to G, A, or S.

In an alternative embodiment of the present disclosure, the C at position 967 in the second peptide fragment is mutated to G.

In an alternative embodiment of the present disclosure, the C at position 967 in the second peptide fragment is mutated to A.

In an alternative embodiment of the present disclosure, the C at position 967 in the second peptide fragment is mutated to S.

According to an embodiment of the present disclosure, the C at position 1060 in the second peptide fragment is mutated to G, A, E, or S.

In an alternative embodiment of the present disclosure, the C at position 1060 in the second peptide fragment is mutated to G.

In an alternative embodiment of the present disclosure, the C at position 1060 in the second peptide fragment is mutated to A.

In an alternative embodiment of the present disclosure, the C at position 1060 in the second peptide fragment is mutated to E.

In an alternative embodiment of the present disclosure, the C at position 1060 in the second peptide fragment is mutated to S.

According to an embodiment of the present disclosure, the C at position 165 in the first peptide fragment is mutated to P; or the C at position 1060 in the second peptide fragment is mutated to E.

Through extensive experiments, the inventors found that, in the process of mutating the above-mentioned positions of the first peptide fragment and the second peptide fragment, the BoNT/A mutants obtained by mutating the above-mentioned mutation positions into amino acid G, A or S have reduced mismatch of disulfide bonds and improved biological activity (virulence) compared to the wild-type BoNT/A, for example, BoNT/A mutant 2 in Table 12 and Table 14. Furthermore, the inventors have also surprisingly found that, through the above-mentioned mutation, by taking the composition of amino acids in the peptide chain close to the mutation position such as amino acid size, hydrophobicity, possible hydrogen bond formation, and charge status into thorough consideration, when cysteine (C165, C1060) with a higher probability of disulfide bond mismatch in wild-type BoNT/A is mutated to P or E, the disulfide bond mismatch can be eliminated to a greater extent or can even be completely eliminated, and the biological activity thereof is also significantly increased by more than 1-fold higher than that of wild-type BoNT/A, such as BoNT/A mutant 1 in Tables 11 and 14.

According to an embodiment of the present disclosure, the first peptide fragment has mutations C134G and C165P compared to the light chain of the wild-type BoNT/A. Thus, the mismatch rate of disulfide bonds in BoNT/A mutants can be further reduced and the biological activity can be increased.

According to an embodiment of the present disclosure, the second peptide fragment has mutations C791A, C967A, and C1060 E compared to the heavy chain of the wild-type BoNT/A. Thus, the mismatch rate of disulfide bonds in BoNT/A mutants can be further reduced and the biological activity can be increased.

According to an embodiment of the present disclosure, the first peptide fragment has mutations C134G and C165P compared to the light chain of a wild-type BoNT/A; and the second peptide fragment has mutations C791A, C967A, and C1060 E compared to the heavy chain of the wild-type BoNT/A. The above-mentioned BoNT/A mutants have higher virulence than the wild-type BoNT/A.

According to an embodiment of the present disclosure, the first peptide fragment has an amino acid sequence as set forth in SEQ ID NO: 3 or SEQ ID NO: 5.

(SEQ ID NO: 3)
MPFVNKQFNYKDPVNGVDIAYIKIPNAGQMQPVKAFKIHNKIWVI
PERDTFTNPEEGDLNPPPEAKQVPVSYYDSTYLSTDNEKDNYLKG
VTKLFERIYSTDLGRMLLTSIVRGIPFWGGSTIDTELKVIDTNGI
NVIQPDGSYRSEELNLVIIGPSADIIQFEPKSFGHEVLNLTRNGY
GSTQYIRFSPDFTFGFEESLEVDTNPLLGAGKFATDPAVTLAHEL
IHAGHRLYGIAINPNRVFKVNTNAYYEMSGLEVSFEELRTFGGHD
AKFIDSLQENEFRLYYYNKFKDIASTLNKAKSIVGTTASLQYMKN
VFKEKYLLSEDTSGKFSVDKLKFDKLYKMLTEIYTEDNFVKFFKV
LNRKTYLNFDKAVFKINIVPKVNYTIYDGFNLRNTNLAANFNGQN
TEINNMNFTKLKNFTGLFEFYKLLCVRGIITSKTKSLDKGYNK;
(SEQ ID NO: 5)
MPFVNKQFNYKDPVNGVDIAYIKIPNAGQMQPVKAFKIHNKIWVI
PERDTFTNPEEGDLNPPPEAKQVPVSYYDSTYLSTDNEKDNYLKG
VTKLFERIYSTDLGRMLLTSIVRGIPFWGGSTIDTELKVIDTNGI
NVIQPDGSYRSEELNLVIIGPSADIIQFEGKSFGHEVLNLTRNGY
GSTQYIRFSPDFTFGFEESLEVDTNPLLGAGKFATDPAVTLAHEL
IHAGHRLYGIAINPNRVFKVNTNAYYEMSGLEVSFEELRTFGGHD
AKFIDSLQENEFRLYYYNKFKDIASTLNKAKSIVGTTASLQYMKN
VFKEKYLLSEDTSGKFSVDKLKFDKLYKMLTEIYTEDNFVKFFKV
LNRKTYLNFDKAVFKINIVPKVNYTIYDGFNLRNTNLAANFNGQN
TEINNMNFTKLKNFTGLFEFYKLLCVRGIITSKTKSLDKGYNK

According to an embodiment of the present disclosure, the second peptide fragment has an amino acid sequence as set forth in SEQ ID NO: 4 or SEQ ID NO: 6.

(SEQ ID NO: 6)
ALNDLCIKVNNWDLFFSPSEDNFTNDLNKGEEITSDTNIEAAEEN
ISLDLIQQYYLTFNFDNEPENISIENLSSDIIGQLELMPNIERFP
NGKKYELDKYTMFHYLRAQEFEHGKSRIALTNSVNEALLNPSRVY
TFFSSDYVKKVNKATEAAMFLGWVEQLVYDFTDETSEVSTTDKIA
DITIIIPYIGPALNIGNMLYKDDFVGALIFSGAVILLEFIPEIAI
PVLGTFALVSYIANKVLTVQTIDNALSKRNEKWDEVYKYIVTNWL
AKVNTQIDLIRKKMKEALENQAEATKAIINYQYNQYTEEEKNNIN
FNIDDLSSKLNESINKAMININKFLNQASVSYLMNSMIPYGVKRL
EDFDASLKDALLKYIYDNRGTLIGQVDRLKDKVNNTLSTDIPFQL
SKYVDNQRLLSTFTEYIKNIINTSILNLRYESNHLIDLSRYASKI
NIGSKVNFDPIDKNQIQLFNLESSKIEVILKNAIVYNSMYENFST
SFWIRIPKYFNSISLNNEYTIINAMENNSGWKVSLNYGEIIWTLQ
DTQEIKQRVVFKYSQMINISDYINRWIFVTITNNRLNNSKIYING
RLIDQKPISNLGNIHASNNIMFKLDGERDTHRYIWIKYFNLFDKE
LNEKEIKDLYDNQSNSGILKDFWGDYLQYDKPYYMLNLYDPNKYV
DVNNVGIRGYMYLKGPRGSVMTTNIYLNSSLYRGTKFIIKKYASG
NKDNIVRNNDRVYINVVVKNKEYRLATNASQAGVEKILSALEIPD
VGNLSQVVVMKSKNDQGITNKCKMNLQDNNGNDIGFIGFHQFNNI
AKLVASNWYNRQIERSSRTLGCSWEFIPVDDGWGERPL(SEQIDN
O:4);ALNDLCIKVNNWDLFFSPSEDNFTNDLNKGEEITSDTNIE
AAEENISLDLIQQYYLTFNFDNEPENISIENLSSDIIGQLELMPN
IERFPNGKKYELDKYTMFHYLRAQEFEHGKSRIALTNSVNEALLN
PSRVYTFFSSDYVKKVNKATEAAMFLGWVEQLVYDFTDETSEVST
TDKIADITIIIPYIGPALNIGNMLYKDDFVGALIFSGAVILLEFI
PEIAIPVLGTFALVSYIANKVLTVQTIDNALSKRNEKWDEVYKYI
VTNWLAKVNTQIDLIRKKMKEALENQAEATKAIINYQYNQYTEEE
KNNINFNIDDLSSKLNESINKAMININKFLNQASVSYLMNSMIPY
GVKRLEDFDASLKDALLKYIYDNRGTLIGQVDRLKDKVNNTLSTD
IPFQLSKYVDNQRLLSTFTEYIKNIINTSILNLRYESNHLIDLSR
YASKINIGSKVNFDPIDKNQIQLFNLESSKIEVILKNAIVYNSMY
ENFSTSFWIRIPKYFNSISLNNEYTIINAMENNSGWKVSLNYGEI
IWTLQDTQEIKQRVVFKYSQMINISDYINRWIFVTITNNRLNNSK
IYINGRLIDQKPISNLGNIHASNNIMFKLDGGRDTHRYIWIKYFN
LFDKELNEKEIKDLYDNQSNSGILKDFWGDYLQYDKPYYMLNLYD
PNKYVDVNNVGIRGYMYLKGPRGSVMTTNIYLNSSLYRGTKFIIK
KYASGNKDNIVRNNDRVYINVVVKNKEYRLATNASQAGVEKILSA
LEIPDVGNLSQVVVMKSKNDQGITNKCKMNLQDNNGNDIGFIGFH
QFNNIAKLVASNWYNRQIERSSRTLGCSWEFIPVDDGWGERPL.

In some alternative embodiments of the present disclosure, the BoNT/A mutant has a first peptide fragment having an amino acid sequence as set forth in SEQ ID NO: 3 and a second peptide fragment having an amino acid sequence as set forth in SEQ ID NO: 4; or the BoNT/A mutant has a first peptide fragment having an amino acid sequence as set forth in SEQ ID NO: 5 and a second peptide fragment having an amino acid sequence as set forth in SEQ ID NO: 6.

### Nucleic Acid Molecules, Expression Vectors, and Genetic Engineering Bacteria

In a second aspect of the present disclosure, a nucleic acid molecule is provided. According to an embodiment of the present disclosure, the nucleic acid molecule encodes a first and/or a second peptide fragment of the BoNT/A mutant according to the first aspect. The nucleic acid molecule of the present disclosure may encode the aforementioned BoNT/A mutant.

According to an embodiment of the present disclosure, the nucleic acid molecule is DNA.

In a third aspect of the present disclosure, an expression vector is provided. According to an embodiment of the present disclosure, the expression vector carries the nucleic acid molecule according to the second aspect. The BoNT/A mutant can be effectively expressed by transforming the expression vector of the present disclosure into the suitable host bacteria under the mediation of a regulatory system, thereby obtaining the BoNT/A mutant according to the first aspect in large quantities.

According to an embodiment of the present disclosure, the expression vector is a plasmid expression vector.

In a fourth aspect of the present disclosure, genetic engineering bacteria are provided. According to an embodiment of the present disclosure, the genetic engineering bacteria include: bacteria carrying the nucleic acid molecule according to the second aspect or the expression vector according to the third aspect; or bacteria expressing the BoNT/A mutant according to the first aspect. The genetic engineering bacteria of the present disclosure can efficiently express the BoNT/A mutant according to the first aspect under suitable conditions.

According to an embodiment of the present disclosure, the genetic engineering bacteria are obtained by transforming the expression vector according to the third aspect into host bacteria.

According to an embodiment of the present disclosure, the host bacteria are E. coli.

### Method for Preparing BoNT/A by Gene Recombination

In a fifth aspect of the present disclosure, a method for preparing BoNT/A by gene recombination is provided. According to an embodiment of the present disclosure, the method includes: performing a first denaturation treatment on a light chain protein to obtain a first denatured product; performing a second denaturation treatment on a heavy chain protein to obtain a second denatured product; and performing a renaturation and assembly treatment on the first denatured product and the second denatured product to obtain the BoNT/A.

By investigating and analyzing the high-order structure and protein characteristics of BoNT/A protein, the inventors have found through extensive experiments that the intact and active BoNT/A can be produced by denaturing the light and heavy chain proteins and then renaturing and assembling them in vitro. Such a method does not require the additional use of protease for toxic activation, thereby avoiding the residues of exogenous tool enzymes and the occurrence of inactive isomer impurities due to incomplete or non-specific cleavage. At the same time, it is not required to separately express the complete BoNT/A protein molecule, thereby avoiding the tendency and risk of the formation of false high-order structure due to the excessively large protein molecule. Therefore, the method can produce a single subtype and single component of BoNT/A. The BoNT/A is free of HA, NTNH, which may be contained in the Clostridium botulinum extract, and also free of non-BoNT/A components such as enzyme residues, which may be produced when activating the toxicity of botulinum toxin using protease subsequent to the expression of the intact BoNT/A protein, thereby having the advantages of batch-to-batch quality stability.

According to an embodiment of the present disclosure, the light chain protein includes the first peptide fragment according to the first aspect or has an amino acid sequence as set forth in SEQ ID NO: 1; and/or the heavy chain protein includes the second peptide fragment according to the first aspect or has an amino acid sequence as set forth in SEQ ID NO: 2.

It is noted that the term “BoNT/A” as used herein may include the wild-type BoNT/A as well as the BoNT/A mutants.

According to an embodiment of the present disclosure, the light chain protein has the amino acid sequence as set forth in SEQ ID NO: 1, and the heavy chain protein has the amino acid sequence as set forth in SEQ ID NO: 2.

According to an embodiment of the present disclosure, the light chain protein or heavy chain protein is obtained by: transforming a plasmid into Escherichia coli, the plasmid carrying a gene encoding the light chain protein or the heavy chain protein; and subjecting the plasmid-transformed Escherichia coli to culturing under conditions suitable for protein expression, inducing, and centrifuging for harvesting bacteria, and bacteria disruption, and centrifuging of disruption product, to obtain the light chain protein or the heavy chain protein. Thus, the light chain protein or the heavy chain protein constituting the BoNT/A protein molecule can be obtained.

According to an embodiment of the present disclosure, the first denaturation treatment and the second denaturation treatment are performed in a denaturation buffer, and the denaturation buffer contains: 5 to 10 M urea, 5 to 15 mM dithiothreitol, and 10 to 30 mM Tris or Tris-HCl. The above-mentioned optimal formulation is obtained by the inventors through extensive experiments, allowing the amino acid chain of the light chain protein and the amino acid chain of the heavy chain protein both to be in a fully extended state.

According to an embodiment of the present disclosure, a pH value of the denaturation buffer ranges from 9.5 to 10.5, which is more favorable to the stretching of the amino acid chains of the light chain protein and the heavy chain protein.

According to an embodiment of the present disclosure, prior to the renaturation and assembly treatment, the first denatured product and the second denatured product are mixed to obtain a mixed denatured solution. The inventors have found through extensive experimentation that the single-chain renaturation and the interchain assembly of heavy and light chains can occur simultaneously by mixing the first and second denatured products first and then simultaneously renaturing them.

According to an embodiment of the present disclosure, in the mixing process, a volume ratio of the first denatured product to the second denatured product is 1:(1-10).

According to an embodiment of the present disclosure, the renaturation and assembly treatment is performed in a renaturation and assembly buffer, and the renaturation and assembly buffer contains: 50 to 150 mM NaCl, 0.1 to 1.0 mM ZnCl₂, 0.1 to 1.0 mM CaCl₂), 1.0 to 10.0 mM reduced glutathione, 1.0 to 10.0 mM oxidized glutathione, 40 to 50 mM Tris-HCl, and 0.4-0.6% Tween 20. The above-mentioned preferred formulation is obtained by the inventors through a large number of screening experiments. The renaturation and assembly buffer enables the denatured light and heavy chain proteins in the stretched state to be folded and assembled. The folding and assembly process can reduce the mismatch rate of disulfide bonds within the light chain protein or the heavy chain protein as well as between the light chain protein and the heavy chain protein, having a higher correct renaturation rate of single chains (light chain and heavy chain) and an assembly efficiency between double chains. In this way, a higher content of the target protein (BoNT/A) can be obtained in the assembly solution, and the subsequently produced BoNT/A protein with the correct intrachain and interchain disulfide bonds can be purer and more active (toxic).

According to an embodiment of the present disclosure, the renaturation and assembly buffer has a pH value ranging from 9.5 to 10.5, which can further improve the correct pairing rate of disulfide bonds of the intrachain of the heavy chain and the intrachain of the light and heavy chains and increase the content of the target protein in the assembly solution.

According to an embodiment of the present disclosure, a volume ratio of the denaturation mixture to the renaturation and assembly buffer is 1:(1-10). The above-mentioned optimal ratio is obtained by the inventors through extensive experiments, and it can increase the correct pairing rate of the disulfide bonds in the heavy chain protein and between the light chain protein and the heavy chain protein during the renaturation and assembly treatment, thereby increasing the content of the target protein in the assembly solution.

According to an embodiment of the present disclosure, the renaturation and assembly treatment is performed for a time period of 12h to 16 h. The above-mentioned optimal renaturation and assembly treatment conditions are obtained by the inventors through extensive experiments, they can facilitate the correct linkage of disulfide bonds in the heavy chain protein and between the light chain protein and the heavy chain protein, enabling assembling to form BoNT/A with the correct high order structure.

According to an embodiment of the present disclosure, the renaturation and assembly treatment is performed under stirring at a rotation speed of 50 rpm to 200 rpm. The above-mentioned optimal renaturation and assembly treatment conditions are obtained by the inventors through extensive experiments, they can facilitate the correct linkage of disulfide bonds in the heavy chain protein and between the light chain protein and the heavy chain protein, enabling assembling to form BoNT/A with the correct high order structure.

According to an embodiment of the present disclosure, the method further includes: sequentially performing hydrophobic chromatography treatment, ammonium sulfate precipitation treatment, dialysis treatment, anion chromatography and molecular exclusion chromatography treatment on an assembly solution obtained from the renaturation and assembly treatment, to obtain the BoNT/A. The above-mentioned purification steps are obtained by the inventors through extensive experiments. The inventors found that, with the above-mentioned purification steps and purification system, impurities in the assembly solution can be advantageously removed, and the purity of the BoNT/A obtained can reach 98.0% or more. Furthermore, the inventors have found that if the sequence of the five above-mentioned purification treatments is changed, or if one or more purification treatments are eliminated, the purity of the final obtained BoNT/A will be significantly reduced.

According to an embodiment of the present disclosure, a mobile phase A1 of the hydrophobic chromatography treatment includes 10-30 mmol/L Tris-HCl, 2-8 mmol/L EDTA, and 1-3 mol/L NaCl; a mobile phase B1 includes 10-30 mmol/L Tris and 2-8 mmol/L EDTA; pH values of the mobile phase A1 and the mobile phase B1 both range from 8.0 to 9.0. The above-mentioned optimal purification conditions are obtained by the inventor through extensive experiments, and they have better purification effect for the assembly solution containing the BoNT/A.

According to an embodiment of the present disclosure, the ammonium sulfate precipitation treatment includes: slowly adding ammonium sulfate to the eluent obtained by the hydrophobic chromatography treatment until reaching a saturated concentration of ammonium sulfate of 80%, stirring at 2-8° C. for 12-24 h to obtain a precipitation liquid, then subjecting the precipitation liquid to centrifugation at 2-8° C. and 10000-15000 rpm for 20-40 min, and discarding the supernatant to obtain a precipitate. The above-mentioned optimal purification conditions are obtained by the inventors through extensive experiments, they can further improve the purity of the BoNT/A.

According to an embodiment of the present disclosure, the dialysis treatment includes: dissolving the precipitate in a first buffer to obtain a to-be-dialyzed solution; performing a first dialysis treatment on the to-be-dialyzed solution and a first dialysate; and performing a second dialysis treatment on a product (retentate) obtained by the first dialysis treatment and a second dialysate. The first buffer is selected from 40-60 mM Tris-HCl buffer. The first dialysate is selected from 40-60 mM Tris-HCl saline dialysate. The second dialysate is selected from 40-60 mM Tris-HCl dialysate. The above-mentioned optimal purification conditions are obtained by the inventor through extensive experiments, and the obtained BoNT/A is high in purity. Furthermore, the inventors have also found through extensive experiments that during the first dialysis treatment, the presence of salt can ensure the continuous solubility of the dissolved protein while avoiding the non-specific adsorption of impurity proteins on the surface of the target protein. Therefore, the selection of a saline dialysate for the first dialysate can further improve the purity of the BoNT/A obtained by the dialysis treatment.

It is noted that “40-60 mM Tris-HCl saline dialysate” refers to a Tris-HCl dialysate containing salt, in which the concentration of Tris-HCl in the dialysate is 40-60 mM.

According to an embodiment of the present disclosure, a ratio of a weight in gram of the precipitate to a volume in milliliter of the first buffer solution is 1:(5-15). The above-mentioned optimal ratio is obtained by the inventor through extensive experiments, and it can further improve the purity of the BoNT/A.

According to an embodiment of the present disclosure, the first dialysis treatment is performed for a dialysis time of 2-5 hours, and the second dialysis treatment is performed for a dialysis time of 12-24 hours. Thus, the effect of the dialysis treatment is better.

According to an embodiment of the present disclosure, the Tris-HCl saline dialysate further includes 200-300 mM NaCl.

According to an embodiment of the present disclosure, a molecular weight cut-off of a dialysis bag for the first dialysis treatment and the second dialysis treatment is from 80 to 120 kDa.

According to an embodiment of the present disclosure, the anion chromatography treatment is selected from DEAE cellulose anion chromatography.

According to an embodiment of the present disclosure, a mobile phase A2 of the DEAE cellulose anion chromatography includes 10-30 mmol/L Tris-HCl; a mobile phase B2 includes 10-30 mmol/L Tris-HCl and 0.5-1.5 mol/L NaCl; the pH values of both the mobile phase A2 and the mobile phase B2 are 8.5. The above-mentioned optimal purification conditions are obtained by the inventors through extensive experiments, and they can further improve the purity of the BoNT/A.

According to an embodiment of the present disclosure, the molecular exclusion chromatography treatment adopts G-25M molecular exclusion chromatography.

According to an embodiment of the present disclosure, a mobile phase C of the G-25M molecular exclusion chromatography includes 10-30 mmol/L Tris-HCl, and the pH of the mobile phase C is 8.0-9.0. The above-mentioned optimal purification conditions are obtained by the inventors through extensive experiments, and they can further improve the purity of the BoNT/A.

According to an embodiment of the present disclosure, the G-25M molecular exclusion chromatography has a loading of ≤30% column volume/cycle and a linear flow rate of 250-350 cm/h.

In a sixth aspect, the present disclosure provides a BoNT/A or a BoNT/A mutant. According to an embodiment of the present disclosure, the BoNT/A is prepared by the method according to the fifth aspect. The inventors have found through experiments that the method described in the fifth aspect can prepare a single subtype and single component BoNT/A with the correct conformation and free of non-BoNT/A components such as HA, NTNH, and enzyme residues, having advantages such as batch-to-batch quality stability.

In a seventh aspect, the present disclosure provides a BoNT/A. According to an embodiment of the present disclosure, the BoNT/A has an LD₅₀ of 1-20 pg/animal and a purity of 98% or more.

According to an embodiment of the present disclosure, the light chain protein of BoNT/A has an amino acid sequence as set forth in SEQ ID NO: 1.

According to an embodiment of the present disclosure, the heavy chain protein of BoNT/A has an amino acid sequence as set forth in SEQ ID NO: 2.

### Pharmaceutical Composition

In an eighth aspect of the present disclosure, a pharmaceutical composition is provided. An embodiment according to the present disclosure, the pharmaceutical composition includes the BoNT/A mutant according to the first aspect, or the BoNT/A prepared by the method according to the fifth aspect. The pharmaceutical composition of the present disclosure has high biological activity (virulence) and can be used not only for medical cosmetology, but also for the treatment or improvement of at least one of strabismus, cervical dystonia, laryngeal dystonia, upper limb focal dystonia, primary hand tremor, sialorrhea, blepharospasm, hemifacial spasm, stroke-caused upper/lower limb spasm, cerebral palsy-caused upper/lower limb spasm, axillary hyperhidrosis, palmar hyperhidrosis, detrusor-sphincter dyssynergia, chronic migraine, and neurogenic and idiopathic overactive bladder.

According to an embodiment of the present disclosure, the medical cosmetology includes ameliorating and/or treating at least one of the following symptoms: frown lines, crow's feet, and forehead wrinkles.

An embodiment according to the present disclosure further includes a pharmaceutically acceptable adjuvant.

According to an embodiment of the present disclosure, the pharmaceutically acceptable adjuvant includes at least one selected from a buffer, a protective agent, an active agent, and an excipient.

It should be noted that a buffer generally refers to a liquid solution having a buffering function, and it should be understood broadly herein. Illustratively, it may be a physiologically compatible buffer system and/or a buffer system composition, which includes, but not limited to, acetic acid, succinic acid, citric acid, histidine, glutamic acid, citrate/acetate, citrate/histidine, succinate/histidine, phosphate, tris buffer systems, and the like.

It should be noted that a protecting agent generally refers to an agent having protecting effects on the pharmaceutical composition, and it should be understood broadly herein. For example, the protecting agent includes, but not limited to, a non-reducing sugar trehalose, sucrose, human albumin, and the like.

According to an embodiment of the present disclosure, the active agent is a non-ionic surfactant.

According to an embodiment of the present disclosure, the non-ionic surfactant includes at least one selected from polysorbate 20, polysorbate 40, polysorbate 60, and polysorbate 80.

According to an embodiment of the present disclosure, the pharmaceutical composition is a liquid composition; the protecting agent includes a non-reducing trehalose and/or sucrose; the non-ionic surfactant includes at least one selected from polysorbate 20, polysorbate 40, polysorbate 60, and polysorbate 80.

According to an embodiment of the present disclosure, the pharmaceutical composition is a lyophilized composition; the protecting agent includes at least one selected from non-reducing trehalose, sucrose, and human albumin; and the excipient is a polyol excipient.

### Uses

In a ninth aspect of the present disclosure, provided is use of the BoNT/A mutant according to the first aspect, the BoNT/A prepared by the method according to the fifth aspect, or the pharmaceutical composition according to the eighth aspect in the preparation of a medicament. The medicament is used in medical cosmetology or in the treatment or amelioration of at least one of strabismus, cervical dystonia, laryngeal dystonia, upper limb focal dystonia, primary hand tremor, sialorrhea, blepharospasm, hemifacial spasm, stroke-caused upper/lower limb spasm, cerebral palsy-caused upper/lower limb spasm, axillary hyperhidrosis, palmar hyperhidrosis, detrusor-sphincter dyssynergia, chronic migraine, and neurogenic and idiopathic overactive bladder.

According to an embodiment of the present disclosure, the medical cosmetology includes wrinkle removal and/or facial slimming.

According to an embodiment of the present disclosure, the medical cosmetology includes ameliorating and/or treating at least one of the following symptoms: frown lines, crow's feet, and forehead wrinkles.

In a tenth aspect of the present disclosure, provided is use of the aforementioned BoNT/A mutant, BoNT/A prepared by the aforementioned method, or the aforementioned pharmaceutical composition in the medical cosmetology or in the treatment or amelioration of a disease. The disease includes at least one of strabismus, cervical dystonia, laryngeal dystonia, upper limb focal dystonia, primary hand tremor, sialorrhea, blepharospasm, hemifacial spasm, stroke-caused upper/lower limb spasm, cerebral palsy-caused upper/lower limb spasm, axillary hyperhidrosis, palmar hyperhidrosis, detrusor-sphincter dyssynergia, chronic migraine, and neurogenic and idiopathic overactive bladder.

According to an embodiment of the present disclosure, the medical cosmetology includes wrinkle removal and/or facial slimming.

According to an embodiment of the present disclosure, the medical cosmetology includes ameliorating and/or treating at least one of the following symptoms: frown lines, crow's feet, and forehead wrinkles.

In an eleventh aspect of the present disclosure, provided is the aforementioned BoNT/A mutant, BoNT/A prepared by the aforementioned method, or the aforementioned pharmaceutical composition for use in the medical cosmetology or in the treatment or amelioration of a disease. The disease includes at least one of strabismus, cervical dystonia, laryngeal dystonia, upper limb focal dystonia, primary hand tremor, sialorrhea, blepharospasm, hemifacial spasm, stroke-caused upper/lower limb spasm, cerebral palsy-caused upper/lower limb spasm, axillary hyperhidrosis, palmar hyperhidrosis, detrusor-sphincter dyssynergia, chronic migraine, and neurogenic and idiopathic overactive bladder.

According to an embodiment of the present disclosure, the medical cosmetology includes wrinkle removal and/or facial slimming.

According to an embodiment of the present disclosure, the medical cosmetology includes ameliorating and/or treating at least one of the following symptoms: frown lines, crow's feet, and forehead wrinkles.

### Method

In a twelfth aspect of the present disclosure, a method for medical cosmetology is provided. According to an embodiment of the present disclosure, the method includes: administrating to a subject the BoNT/A mutant according to the first aspect, the BoNT/A prepared by the method according to the fifth aspect, or the pharmaceutical composition according to the eighth aspect. The method according to the embodiment of the present disclosure can be effectively used in medical cosmetology.

According to an embodiment of the present disclosure, the medical cosmetology includes wrinkle removal and/or facial slimming.

According to an embodiment of the present disclosure, the medical cosmetology includes ameliorating and/or treating at least one of the following symptoms: frown lines, crow's feet, and forehead wrinkles.

According to an embodiment of the present disclosure, the administration includes subcutaneous or intramuscular injection.

In a thirteenth aspect of the present disclosure, a method for ameliorating and/or treating a disease is provided. According to an embodiment of the present disclosure, the method includes: administering to a subject the BoNT/A mutant according to the first aspect, the BoNT/A prepared by the method according to the fifth aspect, or the pharmaceutical composition according to the eighth aspect. The disease is selected from at least one of strabismus, cervical dystonia, laryngeal dystonia, upper limb focal dystonia, primary hand tremor, sialorrhea, blepharospasm, hemifacial spasm, stroke-caused upper/lower limb spasm, cerebral palsy-caused upper/lower limb spasm, axillary hyperhidrosis, palmar hyperhidrosis, detrusor-sphincter dyssynergia, chronic migraine, and neurogenic and idiopathic overactive bladder.

It is noted that, as used herein, a “pharmaceutically acceptable amount” may vary depending on the mode of administration, the severity of the condition to be treated, and the like, with an effective amount being preferred. The selection of a pharmaceutically acceptable amount can be determined by one of ordinary skill in the art based on a variety of factors (e.g., through clinical trials). These factors include, but not limited to, pharmacokinetic parameters of the active ingredient such as bioavailability, metabolism, half-life, and the like; the severity of the disease of the patient to be treated, the weight of the patient, the immune status of the patient, the route of administration, etc. For example, several separate doses may be administered daily or the dose may be proportionally reduced as indicated by the exigencies of the therapeutic situation.

According to an embodiment of the present disclosure, the administration comprises subcutaneous or intramuscular injection.

The solutions of the present disclosure are explained in conjunction with embodiments. Those skilled in the art can understand that the following examples are only used to illustrate the present disclosure, and they should not be considered as limiting the scope of the present disclosure. Where specific techniques or conditions are not specified in the examples, they are performed according to techniques or conditions described in the literature in the art or according to the product specification. The used reagents or instruments without specifying the manufacturer are all conventional and commercially available products.

### Example 1: Design and Construction of Expression Plasmid for Wild-Type BoNT/A Light Chain Protein (BoNT/A-LC) and Heavy Chain Protein (BoNT/A-HC

### 1. Design and Synthesis of Target Gene

Based on the amino acid sequence of the target protein, the nucleotide sequence of the target gene was designed and optimized according to the preferred codons of E. coli to determine the nucleotide sequence of light chain protein and heavy chain protein. The TaKaRa Bio (Dalian) Co. Ltd was entrusted to synthesize the designed nucleotide sequence. The amino acid sequence of light chain protein was set forth in SEQ ID NO: 1; the amino acid sequence of heavy chain protein was set forth in SEQ ID NO: 2; the nucleotide sequence encoding light chain protein was set forth in SEQ ID NO: 7; and the nucleotide sequence encoding heavy chain protein was set forth in SEQ ID NO: 8.

### 2. Construction of Expression Plasmid and Obtaining the Target Genetic Engineering Bacteria.

The two ends of the light chain and heavy chain target genes respectively have XbaI and BamHI digest positions. The target gene sequences of the light and heavy chains and the pET-28a (+) vector were digested with XbaI and BamHI enzymes, respectively. The gel was cut for recovery. The digested target gene fragments were ligated with the digested long fragment of pET-28a (+). After transformation and screening, genetic engineering bacteria expressing the target proteins of the light and heavy chains were obtained.

### Example 2: Preparation of Wild-Type BoNT/A

### 1. Expression of Light Chain Protein and Heavy Chain Protein

The genetic engineering bacteria carrying light and heavy chain genes were inoculated in a shake flask containing LB medium for cultivation, respectively. When the OD₆₀₀ reached 1.6-2.0, the bacteria were transferred into a 5 L fermenter for cultivation. The initial culture volume was 2.5 L, the culture temperature was 37° C., and the rotation speed was 800 rpm. When the OD 600 increased to 30, the bacteria were induced with isopropyl-ß-D-thiogalactoside (IPTG) at a concentration of 0.5 mM for 4-8 h.

The growth of the genetic engineering bacteria in the fermentation broth was microscopically examined to observe the expression status. After protein expression was observed and the induction time was 4-8 h, the fermentation broth was collected and centrifuged at 4° C. and 10000 rpm for 30 min to collect the genetic engineering bacteria.

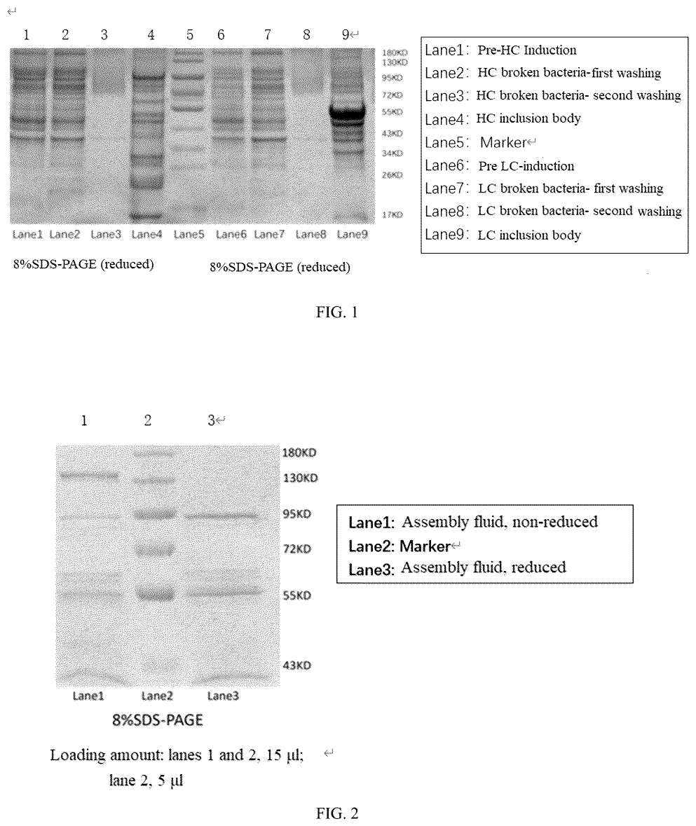

The collected genetic engineering bacteria were subjected to high-pressure homogenization and disruption at a disruption pressure of 700-800 Bar for at least 2 cycles, until no intact cells were observed with the microscopic examination. The inclusion bodies were collected by centrifugation, washed twice with 1:20 (w/v, g/ml) ultra-purified water, respectively. The expression of light chain protein (BoNT/A-LC) and heavy chain protein (BoNT/A-HC) and the SDS-PAGE image of inclusion bodies are shown in FIG. 1.

### 2. Denaturation of BoNT/A-LC Protein, BoNT/A-HC Protein

BoNT/A-LC inclusion bodies and BoNT/A-HC inclusion bodies were weighed respectively according to a weight ratio of 1:4 at room temperature, and dissolved respectively in the denaturation buffer in the proportion of 1:20 (w/v, g/ml). The system was stirred at the rotation speed of 200 rpm until fully dissolved, and the stirring continued for another 60 minutes. The composition of the denaturation buffer included: 8 M urea, 10 mM dithiothreitol (DTT), and 20 mM Tris-HCl, pH 10.0

### 3. Renaturation and In Vitro Assembly of BoNT/A Protein

The light chain denatured product and the heavy chain denatured product obtained in step 2 were mixed and dissolved in an equal volume to obtain a denaturation mixture solution. Then the denaturation mixture solution was mixed with a renaturation and assembly buffer in a volume ratio of 1:10 and continued to stir at a rotation speed of 200 rpm for renaturation and assembly overnight to obtain an assembly solution.

Composition of renaturation and assembly buffer: 100 mM NaCl, 0.5 mM ZnCl₂, 0.5 mM CaCl₂), 5 mM GSH, 5 mM GSSG, 50 mM Tris-HCl, and 0.5% Tween-20, pH 10.0.

A small amount of assembly solution was taken for SDS-PAGE electrophoresis to observe the renaturation and assembly. The results are shown in FIG. 2.

Meanwhile, a gel electrophoresis imager (BIO-RAD, model: ChemiDoc™ MXRS+) Image Lab software was used to scan and analyze the content of the target protein. The analysis results indicate that the content of the target protein (i.e., BoNT/A protein corresponding to No. 4 in Table 1) obtained under this system was 30.2%. During the experiment, in order to optimize the composition of the renaturation and assembly buffer system, a large number of component composition and component concentration exploration experiments, such as metal ions, oxidation-reduction pairs, pH of the system, etc. were conducted. In each experiment, the same amount and the same ratio of BoNT/A-LC protein and BoNT/A-HC protein were controlled to be added to ensure the reliability of the experimental results under the experimental variables. The results are shown in Table 1.

TABLE 1
Influence of different renaturation and assembly system compositions on
renaturation and assembly results

| | | Content of target |
|---|---|---|
| | | protein (BoNT/A) in |
| | | assembly solution |
| No. | Composition of renaturation and assembly system | (%) |
| 1 | 100 mM NaCl, 0.5 mM ZnCl₂, 5 mM GSH, 5 mM | 26.5 |
| | GSSG, 50 mM Tris-HCl, 0.5% Tween-20, pH 10.0 | |
| 2 | 100 mM NaCl, 0.5 mM CaCl₂, 5 mM GSH, 5 mM | 21.8 |
| | GSSG, 50 mM Tris-HCl, 0.5% Tween-20, pH 10.0 | |
| 3 | 100 mM NaCl, 0.5 mM MgCl₂, 5 mM GSH, 5 mM | 29.3 |
| | GSSG, 50 mM Tris-HCl, 0.5% Tween-20, pH 10.0 | |
| 4 | 100 mM NaCl, 0.5 mM ZnCl₂, 0.5 mM CaCl₂, 5 mM | 30.2 |
| | GSH, 5 mM GSSG, 50 mM Tris-HCl, 0.5% Tween-20, | |
| | pH 10.0 | |
| 5 | 100 mM NaCl, 0.5 mM ZnCl₂, 0.5 mM MgCl₂, 5 mM | 28.9 |
| | GSH, 5 mM GSSG, 50 mM Tris-HCl, 0.5% Tween-20, | |
| | pH 10.0 | |
| 6 | 100 mM NaCl, 0.5 mM CaCl2, 0.5 mM MgCl2, 5 mM | 28.2 |
| | GSH, 5 mM GSSG, 50 mM Tris-HCl, 0.5% Tween-20, | |
| | pH 10.0 | |
| 7 | 100 mM NaCl, 0.5 mM ZnCl2, 0.5 mM CaCl2, 0.5 mM | 28.9 |
| | MgCl2, 5 mM GSH, 5 mM GSSG, 50 mM Tris-HCl, | |
| | 0.5% Tween-20, pH 10.0 | |
| 8 | 100 mM NaCl, 0.5 mM ZnCl₂, 0.5 mM CaCl₂, 1 mM | 25.8 |
| | GSH, 5 mM GSSG, 50 mM Tris-HCl, 0.5% Tween-20, | |
| | pH 10.0 | |
| 9 | 100 mM NaCl, 0.5 mM ZnCl₂, 0.5 mM CaCl₂, 5 mM | 27.6 |
| | GSH, 1 mM GSSG, 50 mM Tris-HCl, 0.5% Tween-20, | |
| | pH 10.0 | |
| 10 | 100 mM NaCl, 0.5 mM ZnCl₂, 0.5 mM CaCl₂, 5 mM | 27.2 |
| | GSH, 5 mM GSSG, 50 mM Tris-HCl, 0.5% Tween-20, | |
| | pH 9.0 | |
| 11 | 100 mM NaCl, 0.5 mM ZnCl₂, 0.5 mM CaCl₂, 5 mM | 24.3 |
| | GSH, 5 mM GSSG, 50 mM Tris-HCl, 0.5% Tween-20, | |
| | pH 8.0 | |

### 4. Purification of BoNT/Protein A

The assembly solution obtained in step 3 (the assembly solution No. 4) was subjected to hydrophobic chromatography treatment, ammonium sulfate precipitation treatment, dialysis treatment, DEAE anion chromatography, and molecular exclusion chromatography treatment successively. The specific process was as follows.

Hydrophobic chromatography treatment: the assembly solution obtained in step 3 was added with 4 mol/L NaCl until the final concentration of NaCl in the assembly solution was 2 mol/L, as a loading stock solution. Chromatographic conditions: composition of mobile phase A1: 20 mmol/L Tris+5 mmol/L EDTA+2.0 mol/L NaCl, at pH value of 8.5; composition of mobile phase B1: 20 mmol/L Tris+5 mmol/L EDTA, at pH value of 8.5. Chromatographic steps: mobile phase A1 was equilibrated with 3 column volumes (CV), and loaded to a load capacity. The mobile phase A1 was washed with 6 CV. 0%-100% Mobile phase B1 was eluted in a gradient mode with 10 CV. The samples of more than 200 mAU was collected and absorbed to obtain hydrophobic chromatography eluent.

Ammonium sulfate precipitation treatment: ammonium sulfate was slowly added into the hydrophobic chromatography eluent until reaching a saturation concentration of ammonium sulfate of 80%, and stirred at 4° C. for 20 h to obtain a precipitation liquid. The precipitation liquid was centrifuged at 4° C. and 12,000 rpm for 30 min. The supernatant was discarded to obtain a precipitate.

Dialysis treatment: 50 mM Tris-HCl buffer (pH value 8.5) was taken to dissolve the precipitate according to the proportion of precipitation and buffer solution 1:10 (w/v, g/ml), to obtain a to-be-dialyzed solution. The obtained to-be-dialyzed solution was transferred to a dialysis bag (molecular weight cut-off: 100 kDa), stirred (100 rpm) for 3 h at 4° ° C. with 50 mM Tris-HCl buffer (containing 100 mM NaCl, pH 8.5) having a volume 20 times that of the to-be-dialyzed solution, which was further dialyzed for 24 h with stirring (100 rpm) with 50 mM Tris-HCl buffer (pH 8.5) having a volume 20 times that of the to-be-dialyzed solution.

DEAE anion chromatography: the dialysate obtained from the dialysis treatment was directly loaded into chromatography. Composition of mobile phase A2: 20 mmol/L Tris, pH 8.5; composition of mobile phase B2: 20 mmol/L Tris+1.0 mol/L NaCl, pH 8.5. Chromatographic steps: mobile phase A2 was equilibrated with 3 CV, and loaded to a load capacity. Mobile phase A2 was washed with 3 CV. 0%-50% Mobile phase B1 was eluted in a gradient mode with 20 CV to obtain an eluent.

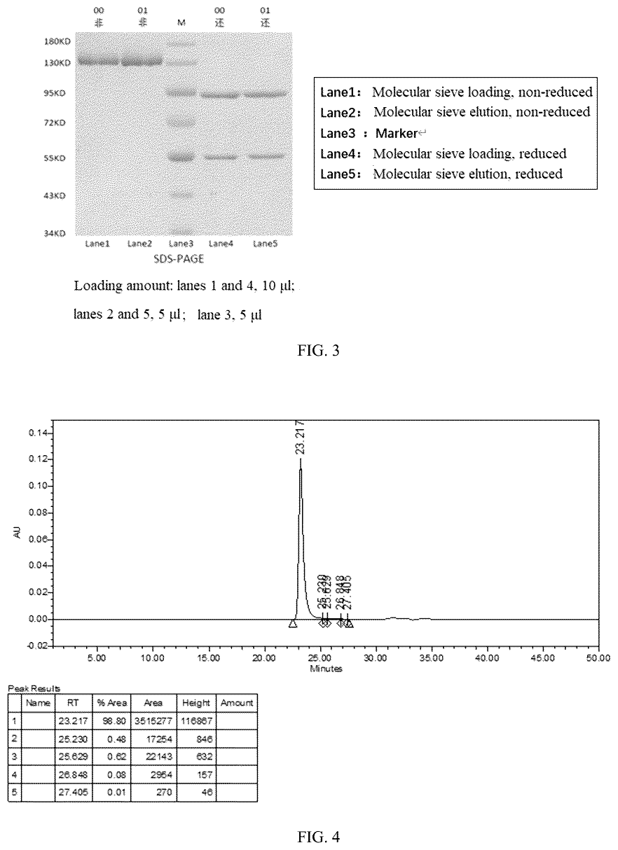

G-25M molecular exclusion chromatography: the eluate obtained in the DEAE anion chromatography treatment was directly loaded to molecular exclusion chromatography treatment. Mobile phase C: 20 mmol/L Tris, pH 8.5, loading≤30% column volume/cycle, linear flow rate 300 cm/h. Each elution peak was collected, and the combined sample was detected by SDS-PAGE for purity, so as to obtain the BoNT/A sample. The SDS-PAGE electrophoresis result of the sample is as shown in FIG. 3. The target protein (i.e., the BoNT/A protein prepared corresponding to No. 6 in Table 2) had an SEC chromatographic purity of 98.8% (FIG. 4).

In order to obtain the above-mentioned optimized purification process, the inventors conducted a large number of experiments in terms of influence factors, in order to optimize the purification process and obtain a high-purity BoNT/A sample, focusing on the composition of purification unit operation and the sequence of purification unit operation. Each experimental process and each unit operation are performed approximately under the same experimental conditions, including, but not limited to, the composition of loading buffer, elution conditions, pH value, filler capacity, loading flow rate, column height, column diameter, column volume, etc. The experimental results are shown in Table 2.

TABLE 2
Influence of purification process on purity of target protein

| | | Purity of target |
|---|---|---|
| No. | Purification process/step | protein (%) |
| 1 | Ammonium sulfate precipitation treatment, hydrophobic | 83.7 |
| | chromatography treatment, DEAE anion chromatography, | |
| | pH 8.5 | |
| 2 | Ammonium sulfate precipitation treatment, DEAE anion | 85.8 |
| | chromatography, hydrophobic chromatography treatment, | |
| | pH 8.5 | |
| 3 | Hydrophobic chromatography treatment, ammonium sulfate | 92.7 |
| | precipitation treatment, DEAE anion chromatography, pH | |
| | 8.5 | |
| 4 | Hydrophobic chromatography treatment, ammonium sulfate | 98.5 |
| | precipitation treatment, DEAE anion chromatography, | |
| | dialysis treatment, pH 8.5 | |
| 5 | Hydrophobic chromatography treatment, ammonium sulfate | 98.5 |
| | precipitation treatment, dialysis treatment, DEAE anion | |
| | chromatography, pH 8.5 | |
| 6 | Hydrophobic chromatography treatment, ammonium sulfate | 98.8 |
| | precipitation treatment, dialysis treatment, DEAE anion | |
| | chromatography, molecular exclusion chromatography | |
| | treatment, pH 8.5 | |
| 7 | Hydrophobic chromatography treatment, ammonium sulfate | 95.2 |
| | precipitation treatment, dialysis treatment, DEAE anion | |
| | chromatography, molecular exclusion chromatography | |
| | treatment, pH 7.5 | |

### Example 3: Structural Characterization of BoNT/A Protein

1. Complete molecular weight detection

1.1. Sample Processing: 1 ml of the BoNT/A protein sample prepared in Example 2 was concentrated 5-fold and mixed evenly for loading.

1.2. UPLC conditions

Chromatographic column: BioResolve RP mAb 2.7 μm, 2.1 mm×100 mm, Waters 01093809916819; column temperature: 50° ° C.; detection wavelength: 280 nm; flow rate: 0.3 ml/min; loading amount: 10 μl.

Mobile phase A: 0.05% TFA.H₂O (aqueous trifluoroacetic acid); mobile phase B: 0.05% trifluoroacetic acid-acetonitrile (TFA-CAN).

Gradient:

| Time (min) | 0 | 12 | 13 | 15 | 16 | 20 |
|---|---|---|---|---|---|---|
| B | 10 | 60 | 90 | 90 | 10 | 10 |

1.3. MS conditions

Ionization mode: ESI positive; mass scan range: 300-4000 Da; capillary voltage: 3.0 KV; source temperature: 100° C.; cone voltage: 150 KV; de-solvation gas temperature: 450° C.; cone blowback gas flow rate: 50 L/H; de-solvation gas flow rate: 800 L/H.

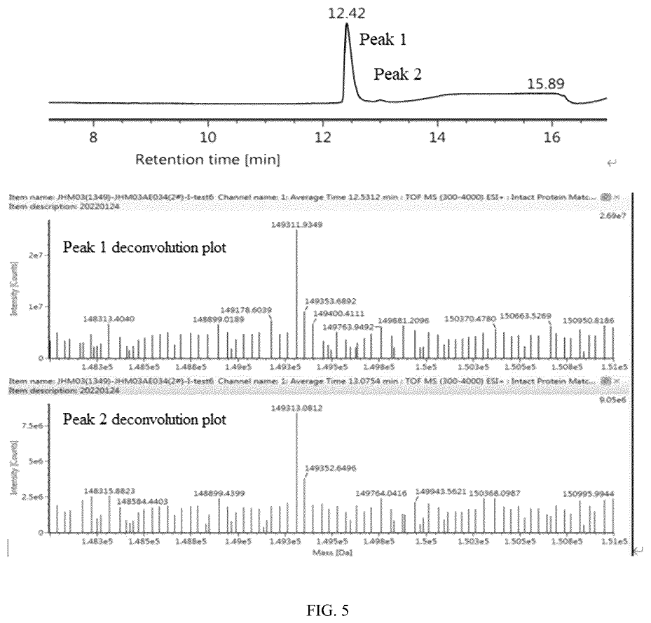

1.4. Data collection and processing: data collection was performed using Masslynx V4.1 software, and data analysis was performed by means of UNIFI software. The analysis results can refer to Table 3 and FIG. 5.

TABLE 3
Complete molecular weight data analysis

| | | Theoretical | Measured | | |
|---|---|---|---|---|---|
| | Identified | molecular | molecular | Error | |
| Peak | component | weight (Da) | weight (Da) | (ppm) | Proportion |
| 1 | BONT/A | 149306.7636 | 149311.9349 | 34.6 | 97.2% |
| 2 | Bont/A | 149308.7795 | 149313.0812 | 28.8 | 2.8% |
| | (deamidation | | | | |
| | modified) | | | | |

2. Detection of reduced molecular weight

2.1. Sample Processing: 150 μl of BoNT/A protein sample prepared in Example 2 was added with 150 μl of 7 mol/L guanidine hydrochloride, 0.1 mol/L Tris (pH value 8.0) and 3 μl of 1 mol/L DTT, incubated at 70° ° C. for 30 min, and mixed evenly.

2.2. UPLC conditions were identical to step 1.2.

2.3. The MS conditions were the same as in Step 1.3, except that the cone voltage was 40 KV.

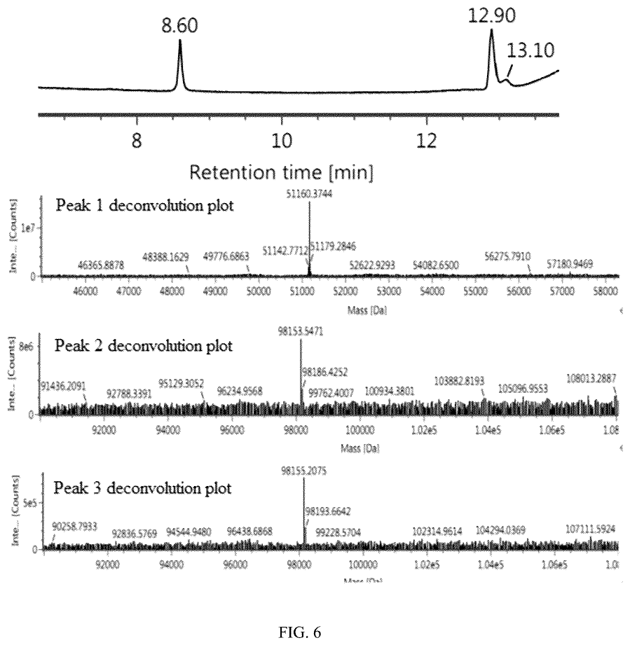

2.4. Data collection and processing: data collection was performed using Masslynx V4.1 software and data analysis was performed by means of UNIFI software. The analysis results can refer to Table 4 and FIG. 6.

TABLE 4
Data analysis of reduced molecular weight

| | | Theoretical | Measured | |
|---|---|---|---|---|
| | | molecular | molecular | Error |
| Peak | Identified component | weight (Da | weight (Da) | (ppm) |
| 1 | BONT/A-LC | 51159.5785 | 51160.3744 | 15.6 |
| 2 | BONT/A-HC | 98151.2169 | 98153.5471 | 28.8 |
| 3 | Bont/A-HC | 98152.3632 | 98155.2075 | 30.0 |
| | (deamidation modified) | | | |

3. Analysis of disulfide bond

3.1. Sample processing: 1 ml of BoNT/A protein sample prepared in Example 2 was taken and subjected to 5-fold concentration. 350 μl of 0.05 mol/L ammonium bicarbonate was added into each concentration tube, mixed evenly, and concentrated to 100 μl. 4 μl of 1 mol/L iodoacetamide solution (IAM) and 350 μl of 0.05 mol/L ammonium bicarbonate were added into each concentration tube, mixed evenly, and concentrated to 100 μl. 180 μl of the concentrated sample was taken and added with 20 μl of 1% RapiGest SF surfactant. The mixture was incubated for 30 min at 60° C., added with 8 μg of trypsin, incubated overnight at 37° C. Thereafter, the mixture was taken out, added with 1 μl of formic acid, incubated 45 min at 37° C. Thereafter, the mixture was taken out for 10 min of centrifugation at 13,000 rpm. The supernatant was mixed evenly for loading.

3.2. UPLC conditions

Chromatographic column: UPLC BEH C18 1.7 μm, 2.1 mm×150 mm, Waters 01443804318321; column temperature: 60° C.; detection wavelength: 215 nm; flow rate: 0.3 ml/min; loading amount: 10 μl.

Mobile phase A: 0.05% TFA.H₂O; and mobile phase B: 0.05% TFA-ACN.

Gradient:

| Time (min) | 0 | 5 | 140 | 141 | 145 | 146 | 150 |
|---|---|---|---|---|---|---|---|
| B | 2 | 2 | 40 | 100 | 100 | 2 | 2 |

3.3. MS conditions

Ionization mode: ESI positive; mass scan range: 100-2,000 Da; capillary voltage: 3.0 KV; source temperature: 100° C.; cone voltage: 40 KV; de-solvation gas temperature: 450° C.; cone blowback gas flow rate: 50 L/H; de-solvation gas flow rate: 800 L/H.

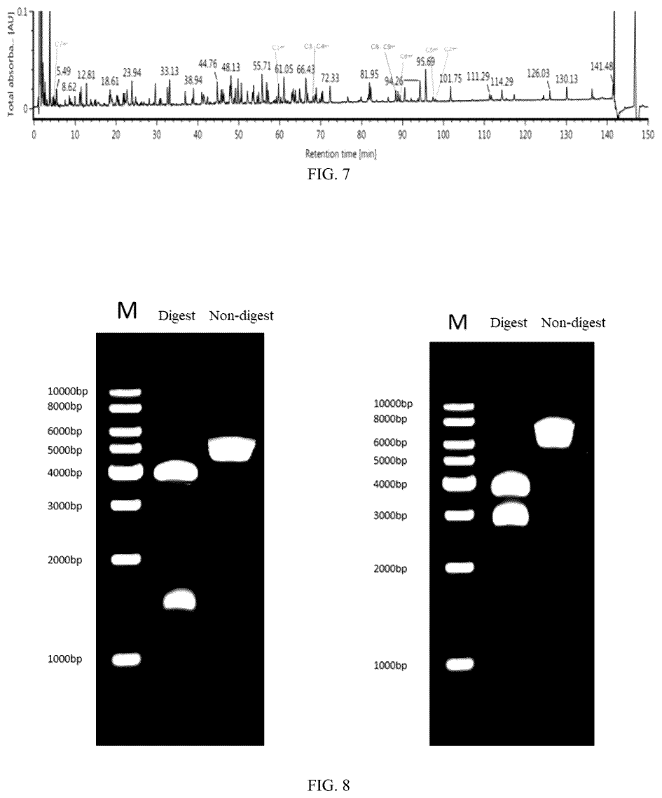

3.4. Data collection and processing: data collection was performed using Masslynx V4.1 software and data analysis was performed by means of UNIFI software. The analysis results can refer to Table 5 and FIG. 7.

TABLE 5
Data analysis of disulfide bond

| | | | Theoretical | Measured | |
|---|---|---|---|---|---|
| Cysteine | | | molecular | molecular | Error |
| position | Structure | Identified peptide fragment | weight (Da) | weight (Da) | (ppm) |
| C3-C4 | Disulfide bond | LLCVR═ALNDLCIK | 1489.8229 | 1489.8216 | 0.9 |
| C8-C9 | Disulfide bond | CK═TLGCSWEFIPVDDG | 2524.1635 | 2524.1568 | 2.7 |
| | | WGERPL | | | |
| C1 | Free sulfhydryl | VIDTNCINVIQPDGSYR | 1963.9542 | 1963.9492 | 2.5 |
| C2 | Free sulfhydryl | SEELNLVIIGPSADIIQFE | 2375.2163 | 2375.2229 | 2.8 |
| | | CK | | | |
| C5 | Free sulfhydryl | FLNQCSVSYLMNSMIPY | 2351.1233 | 2351.1138 | 4.0 |
| | | GVKR | | | |
| C6 | Free sulfhydryl | YFNSISLNNEYTIINCME | 2911.3026 | 2911.2913 | 3.9 |
| | | NNSGWK | | | |
| C7 | Free sulfhydryl | LDGCR | 620.2821 | 620.2825 | 0.7 |

4. Data analysis results

The analysis data in Table 3 to Table 5 reveal that the complete molecular weight, reduced molecular weight, and disulfide linkage of BoNT/A protein were consistent with the theoretical values, indicating that the purified BoNT/A protein has the correct sequence and the correct high-order conformation.

### Example 4: Preliminary Determination of In Vivo Activity (Toxicity) of BoNT/A Protein

Experimental Animals: ICR mice, 4-5 weeks old, 17-22 g.

Different batches of BoNT/A samples prepared according to the method of Example 2 were selected as test samples, numbered 1 #, 2 #, 3 #, and 4 #, respectively. Each test sample had a volume of 0.5 ml, which was then added with 4.5 ml of normal saline for 10-fold gradient dilution, shaken 3-4 times, and mixed well. The diluted samples were placed into an ice-water mixture. Four test samples were inoculated into four groups of mice (5 mice in each group) by intraperitoneal inoculation. Each mouse was inoculated with 0.1 ml. The animals were observed for 3 consecutive days. The death of the animals was recorded daily. The analysis results can refer to Table 6.

TABLE 6
Death of animals in different groups

| Date of | Observation | | | | | |
|---|---|---|---|---|---|---|
| experiment | time | Lot: 1# | | | | |
| 0 h | 10:00 | — | — | — | — | — |
| 6 h | 16:00 | Incidence, | Incidence, | Incidence, | — | — |
| | | tachypnea, | tachypnea, | tachypnea, | | |
| | | delayed action | delayed action | delayed action | | |
| 22 h | 8:30 | + Death | + Death | + Death | + Death | + Death |

| Date of | Observation | | | | | |
|---|---|---|---|---|---|---|
| experiment | time | Lot: 2# | | | | |
| 0 h | 10:00 | — | — | — | — | — |
| 6 h | 16:00 | + Death | + Death | + Death | + Death | + Death |
| Date of | Observation | | | | | |
| experiment | time | Lot: 3# | | | | |
| 0 h | 10:00 | — | — | — | — | — |
| 6 h | 16:00 | + Death | + Death | + Death | + Death | + Death |
| Date of | Observation | | | | | |
| experiment | time | Lot: 4# | | | | |
| 0 h | 10:00 | — | — | — | — | — |
| 6 h | 16:00 | + Death | + Death | + Death | + Death | + Death |

Note:
“—” indicates that the experimental animal is healthy and alive, and “+” indicates that the experimental animal is dead, as specified by the abnormality of the experimental animal.

The experimental results reveal that: at the given test sample concentrations, each batch of test sample (1 #, 2 #, 3 #, 4 #) showed good in vivo activity (toxicity), and the test samples 2 #, 3 #, 4 # had relatively better in vivo activity (toxicity).

### Example 5: Virulence Assay for BoNT/A Protein

Sixty ICR mice (30 ♂ 30 ♀) were weighed and randomly divided into 10 groups with 6 mice in each group (3 ♂ 3 ♀). The BoNT/Protein A prepared as described in Example 2 was used as the test sample, starting with a dose of 300 pg/animal and setting 10 dose gradients in a 2-fold gradient: 300, 150, 75, 37.5, 18.75, 9.375, 4.6875, 2.34375, 1.171875 and 0.5859375 pg/animal. Each mouse in the groups was administered with the test sample at the corresponding concentration. Each mouse was intraperitoneally injected with 0.1 ml of the BoNT/A protein prepared according to the method of Example 2. The number of deaths in each group was observed and recorded daily for 4 consecutive days. LD₅₀ values were calculated with GraphPad 26 by logistic regression fitting of transformed dose log and mortality. The results reveal that the LD₅₀ of the test sample was 4.023 pg/animal, and the corrected sample virulence was 1.06×10⁸ LD₅₀/mg. The analysis results can refer to Table 7.

TABLE 7
Animal mortality at gradient concentrations

| Loading | Intragroup | Intragroup | Intragroup | Intragroup | | | |
|---|---|---|---|---|---|---|---|
| amount | death on | death on | death on | death on | Total | Total | |
| (pg) | day 1 | day 2 | day 3 | day 4 | deaths | animals | Mortality |
| 300 | 6 | 6 | 6 | 6 | 6 | 6 | 100% |
| 150 | 6 | 6 | 6 | 6 | 6 | 6 | 100% |
| 75 | 6 | 6 | 6 | 6 | 6 | 6 | 100% |
| 37.5 | 6 | 6 | 6 | 6 | 6 | 6 | 100% |
| 18.75 | 4 | 6 | 6 | 6 | 6 | 6 | 100% |
| 9.375 | 0 | 5 | 5 | 5 | 5 | 6 | 83.3% |
| 4.6875 | 0 | 3 | 4 | 4 | 4 | 6 | 66.7% |
| 2.34375 | 0 | 1 | 1 | 1 | 1 | 6 | 16.7% |
| 1.171875 | 0 | 0 | 0 | 0 | 0 | 6 | 0% |
| 0.5859375 | 0 | 0 | 0 | 0 | 0 | 6 | 0% |

### Example 6: Construction of Genetic Engineering Bacteria Expressing Light and Heavy Chain Mutants of BoNT/A Mutant 1

The nucleotide sequence (amino acid sequence SEQ ID NO: 3; nucleotide sequence SEQ ID NO: 9) encoding the light chain mutant of BoNT/A mutant 1, and the nucleotide sequence (amino acid sequence SEQ ID NO: 4; nucleotide sequence SEQ ID NO: 10) encoding the heavy chain mutant of BoNT/A mutant 1 were supplemented with XbaI and BamHI cleavage positions at both ends, respectively. The sequences were synthesized by TaKaRa Bio (Dalian) Co. Ltd to obtain the target gene nucleotide sequences of light chain mutant and heavy chain mutant, respectively.

The nucleotide sequences of the target genes of the light chain mutant and the heavy chain mutant were double-digest with XbaI and BamHI, respectively, and the vector pET-5a (+) was also double-digest with XbaI and BamHI, respectively. The target gene and the target fragments obtained after double-digestion were subjected to gel cutting for recovery. The fragments, obtained by the double-digestion, of the light chain mutant and the heavy chain mutant were respectively ligated with the fragment, obtained by the double-digestion, of the vector. The ligation system was subjected to transformation, target protein clone screening, and plasmid digestion verification to finally obtain the genetic engineering bacteria expressing the light chain mutant protein and the heavy chain mutant protein of BoNT/A. FIG. 8 illustrates the plasmid validation of genetic engineering bacteria for light chain mutant protein and heavy chain mutant protein, where double digestion agarose electrophoresis for light chain mutant plasmid is illustrated on the left and double digestion agarose electrophoresis for heavy chain mutant plasmid is illustrated on the right.

The nucleotide sequence encoding the light chain mutant of BoNT/A mutant 1 is set forth in SEQ ID NO: 9:

(SEQ ID NO: 9)
ATGCCGTTTGTGAACAAACAGTTTAACTATAAAGATCCGGTGAAC
GGCGTGGATATTGCGTATATTAAAATTCCGAACGCGGGTCAGATG
CAGCCGGTGAAAGCGTTTAAAATTCATAACAAAATTTGGGTGATT
CCGGAACGCGATACCTTTACCAACCCGGAAGAAGGCGATCTGAAC
CCGCCACCGGAAGCGAAACAAGTGCCGGTGAGCTATTATGATAGC
ACCTATCTGAGCACCGATAACGAAAAAGATAACTATCTGAAAGGC
GTGACCAAACTGTTTGAACGCATTTATAGCACCGATCTGGGCCGC
ATGCTGCTGACGAGCATTGTGCGCGGCATTCCGTTCTGGGGCGGC
AGCACCATTGATACCGAACTGAAAGTGATTGATACCAACGGTATT
AACGTGATTCAGCCGGATGGCAGCTATCGCAGCGAAGAACTGAAC
CTGGTGATTATTGGCCCGAGCGCGGATATTATTCAGTTTGAACCT
AAAAGCTTTGGCCATGAAGTGCTGAACCTGACCCGCAACGGCTAT
GGCAGCACGCAGTATATTCGCTTTAGCCCGGATTTTACCTTTGGC
TTTGAAGAAAGCCTGGAAGTGGATACCAACCCGCTGCTGGGCGCG
GGCAAATTTGCGACCGATCCGGCGGTGACCCTGGCGCATGAACTG
ATTCATGCGGGCCATCGCCTGTATGGCATTGCGATTAACCCGAAC
CGCGTGTTTAAAGTGAACACCAACGCGTATTATGAAATGAGCGGC
CTGGAAGTGAGCTTTGAAGAACTGCGCACCTTTGGCGGCCATGAT
GCGAAATTTATTGATAGCCTGCAAGAAAACGAATTTCGCCTGTAT
TACTATAACAAATTTAAAGATATTGCGAGCACCCTGAACAAAGCG
AAAAGCATTGTGGGCACCACCGCGAGCCTGCAGTATATGAAAAAC
GTGTTTAAAGAAAAATATCTGCTGAGCGAAGATACGAGCGGCAAA
TTTAGCGTGGATAAACTGAAATTTGATAAACTGTATAAAATGCTG
ACCGAAATTTATACCGAAGATAACTTTGTGAAATTTTTTAAAGTG
CTGAACCGCAAGACCTATCTGAACTTTGATAAAGCGGTGTTTAAA
ATTAACATTGTGCCGAAAGTGAACTATACCATTTATGATGGCTTT
AACCTGCGCAACACCAACCTGGCGGCGAACTTTAACGGTCAGAAC
ACCGAAATTAACAACATGAACTTTACCAAACTGAAAAACTTTACC
GGCCTGTTTGAATTTTATAAACTGCTGTGCGTTCGCGGCATCATT
ACGAGCAAAACCAAAAGCCTGGATAAAGGCTATAACAAATAA;

The nucleotide sequence encoding the heavy chain mutant of BoNT/A mutant 1 is set forth in SEQ ID NO: 10:

(SEQ ID NO: 10)
ATGGCGCTGAACGATCTGTGCATTAAAGTGAATAATTGGGATCTG
TTTTTTAGCCCGAGCGAAGATAACTTTACCAACGATCTGAACAAA
GGCGAAGAAATTACGAGCGATACCAACATTGAAGCGGCGGAAGAG
AACATTAGTCTGGATCTGATTCAGCAGTATTATCTGACCTTTAAC
TTTGATAACGAACCGGAAAACATTAGTATTGAAAACCTGAGCAGC
GATATTATTGGTCAGCTGGAACTGATGCCGAACATTGAACGCTTT
CCGAACGGCAAAAAATATGAACTGGATAAATATACCATGTTTCAT
TATCTGCGCGCGCAAGAATTTGAACATGGCAAAAGCCGCATTGCG
CTGACCAACAGCGTGAACGAAGCGCTGCTGAACCCGAGCCGCGTG
TATACCTTTTTTAGCAGCGATTATGTGAAAAAAGTGAACAAAGCG
ACCGAAGCGGCGATGTTTCTGGGCTGGGTGGAACAGCTGGTGTAT
GATTTTACCGATGAGACGAGCGAAGTGAGTACCACCGATAAAATT
GCGGATATTACCATTATCATTCCGTATATTGGCCCGGCGCTGAAC
ATTGGCAACATGCTGTATAAAGATGATTTTGTGGGCGCGCTGATT
TTTAGCGGCGCGGTGATTCTGCTGGAATTTATTCCGGAAATCGCG
ATTCCGGTGCTGGGCACCTTTGCGCTGGTGAGCTATATTGCGAAC
AAAGTGCTGACCGTGCAGACCATTGATAACGCGCTGAGCAAACGC
AACGAAAAATGGGATGAAGTGTATAAATATATTGTGACCAACTGG
CTGGCGAAAGTGAACACGCAGATTGATCTGATTCGCAAAAAAATG
AAAGAAGCGCTGGAAAACCAAGCGGAAGCGACCAAGGCGATTATT
AACTATCAGTATAATCAGTATACCGAAGAGGAAAAAAACAACATT
AACTTTAACATTGATGATCTGAGCAGCAAATTAAATGAAAGCATT
AACAAAGCGATGATCAACATTAACAAGTTTCTGAATCAGGCAAGC
GTGAGCTATCTGATGAACAGCATGATTCCGTATGGCGTGAAACGC
CTGGAAGATTTTGATGCGAGCCTGAAAGATGCGCTGCTGAAATAT
ATTTATGATAACCGCGGCACCCTGATTGGCCAAGTGGATCGCCTG
AAAGATAAAGTTAATAACACGCTGAGCACCGATATTCCGTTTCAG
CTGAGCAAATATGTGGATAATCAGCGCCTGCTGAGCACCTTTACC
GAATATATTAAAAACATTATTAACACGAGCATTCTGAACCTGCGC
TATGAAAGCAACCATCTGATTGATCTGAGCCGCTATGCGAGCAAA
ATTAACATTGGCAGCAAAGTGAACTTTGATCCGATTGATAAAAAT
CAGATTCAGCTGTTTAACCTGGAAAGCAGCAAAATTGAAGTGATT
CTGAAAAACGCGATTGTGTATAACAGCATGTATGAAAACTTTAGC
ACGAGCTTTTGGATTCGCATTCCGAAATACTTTAACAGCATCAGC
CTGAACAACGAATATACCATTATTAACGCAATGGAAAACAACAGC
GGCTGGAAAGTGAGCCTGAACTATGGCGAAATTATTTGGACCCTG
CAAGATACCCAAGAAATTAAACAGCGCGTGGTGTTTAAATATAGT
CAGATGATTAACATTAGCGATTATATTAACCGCTGGATTTTTGTG
ACCATTACCAACAACCGTCTGAACAACAGCAAAATTTATATTAAC
GGCCGCCTGATTGATCAGAAACCGATTAGCAACCTGGGCAACATT
CATGCGAGCAACAACATTATGTTTAAACTGGATGGCGAGCGCGAT
ACGCATCGCTATATCTGGATTAAATATTTTAATCTGTTCGACAAA
GAACTGAACGAAAAAGAAATTAAAGATCTGTATGATAATCAGAGC
AACAGCGGCATTCTGAAAGATTTTTGGGGCGATTATCTGCAGTAT
GATAAACCGTATTATATGCTGAACCTGTATGATCCGAACAAATAT
GTGGATGTGAACAACGTGGGCATTCGCGGCTATATGTATCTGAAA
GGCCCGCGCGGCAGCGTGATGACCACCAACATTTATCTGAACAGC
AGCCTGTATCGCGGCACCAAATTTATTATTAAAAAATATGCGAGC
GGCAACAAAGATAACATTGTGCGCAACAACGATCGCGTGTATATT
AACGTGGTTGTGAAAAACAAAGAATATCGCCTGGCGACCAACGCG
AGCCAAGCGGGCGTGGAAAAAATTCTGAGCGCGCTGGAAATTCCG
GATGTGGGCAACCTGAGCCAAGTGGTTGTGATGAAAAGCAAAAAC
GATCAAGGCATTACCAACAAGTGCAAAATGAACCTGCAAGATAAC
AACGGCAACGATATTGGCTTTATTGGCTTTCATCAGTTTAACAAC
ATTGCGAAACTGGTGGCGAGCAACTGGTATAACCGTCAGATTGAA
CGCAGCAGCCGCACCCTGGGCTGCAGCTGGGAATTTATTCCGGTT
GATGATGGCTGGGGCGAACGCCCGCTGTAA.

### Example 7: Preparation of BoNT/A Mutant 1

The genetic engineering bacteria expressing the light and heavy chain mutant proteins, as prepared in example 6, were cultured, respectively. Through fermentation expression, denaturation and renaturation, in vitro assembly and purification, the BoNT/A mutant 1 (also referred to as BoNT/A mutant protein 1 in the present example, or mutant 1) was obtained. Compared with the wild-type BoNT/A, the mutation positions of mutant 1 are C134G, C165P, C791A, C967A, and C1060E. The specific steps are described below.

### 1. Fermentation Expression of Light Chain Mutant Protein and Heavy Chain Mutant Protein

The genetic engineering bacteria expressing the light chain mutant protein and the heavy chain mutant protein, as prepared in Example 6, were respectively inoculated into a shake flask filled with LB medium for cultivation. When the OD₆₀₀ reached 1.6-2.0, they were transferred into a 5 L fermenter for cultivation. The initial cultivation volume was 2.5 L; the cultivation temperature was 37° C.; and the rotation speed was 800 rpm. When the OD₆₀₀ increased to 30, the induction was performed with isopropyl thiogalactoside (IPTG), as an inducer, at a concentration of 0.5 mM for an induction time of 4-8 h.

The growth of the genetic engineering bacteria in the fermentation broth was microscopically examined to observe the expression status. After protein expression was observed and the induction had last for 4-8 h, the fermentation culturing was ended, and the fermentation broth was collected and centrifuged at the rotation speed of 10,000 rpm and the temperature of 4° C. to collect the bacteria. The collected bacteria were observed by means of the optical microscope to determine whether the bacteria form was the typical form of Escherichia coli and whether there was no other microbial contamination.

The collected bacteria were subjected to high-pressure homogenization and disruption at a disruption pressure of 700-800 Bar for at least 2 cycles until no intact cells were observed under microscopy. The inclusion bodies were collected by centrifugation. The inclusion bodies were washed twice with 1:20 (w/v, g/ml) ultra-clean water to obtain light chain mutant inclusion body protein (abbreviation for light chain mutant inclusion body) and heavy chain mutant inclusion body protein (abbreviation for heavy chain mutant inclusion body).

### 2. Denaturation of Light Chain Mutant Protein and Heavy Chain Mutant Protein

At room temperature, light chain mutant inclusion bodies and heavy chain mutant inclusion bodies were weighed, respectively, according to a weight ratio of light chain mutant inclusion bodies to heavy chain mutant inclusion bodies of 1:4 (g/g). Then, the light chain mutant inclusion bodies or the heavy chain mutant inclusion bodies were dissolved in the denaturation buffer (20 mM Tris containing 8 M urea and 10 mM DTT) with a pH value of 10.0 respectively according to the weight-to-volume ratio of 1:20 (w/v, g/ml) of the light chain mutant inclusion bodies or the heavy chain mutant inclusion bodies to the denaturation buffer, and stirred at a rotation speed of 200 rpm until they were completely dissolved, thereby obtaining the denaturation solution (i.e., the denatured product of the light chain mutant or the denatured product of the heavy chain mutant).

### 3. Renaturation and In Vitro Assembly of Light Chain Mutant Proteins and Heavy Chain Mutant Proteins

The denatured product of the light chain mutant and the denatured product of the heavy chain mutant, which were obtained in step (2), were mixed and dissolved in equal volumes to obtain a denaturation mixture solution. Then, the denaturation mixture solution was mixed with a renaturation and assembly buffer in a volume ratio of 1:10, and the mixture was stirred at a rotation speed of 200 rpm, and subjected to renaturation and assemble overnight to obtain an assembly solution.

Composition of renaturation and assembly buffer: 100 mM NaCl, 0.5 mM ZnCl₂, 0.5 mM CaCl₂, 5 mM GSH, 5 mM GSSG, 50 mM Tris-HCl, and 0.5% Tween-20, pH 10.0.

A small amount of the assembly solution was concentrated at a volume ratio of 5:1 (ml/ml), and the assembly was observed by means of SDS-PAGE electrophoresis. When a single band of 150 KD was observed through the SDS-PAGE electrophoresis, the assembly was stopped to proceed to downstream purification.

### 4. Purification of BoNT/A Mutant Protein

Hydrophobic chromatography treatment, ammonium sulfate precipitation treatment, dialysis treatment, DEAE anion chromatography and molecular exclusion chromatography treatment were successively performed to obtain BoNT/A mutant 1.

Hydrophobic chromatography treatment: 4 mol/L NaCl was added to the above-mentioned solution for in vitro assembly until the final concentration of NaCl was 2 mol/L, as the loading stock solution. The mobile phase A was equilibrated with 3 CV, and loaded to a load capacity. The mobile phase A was washed with 6 CV, and 0% B to 100% B was eluted in a gradient mode with 10 CV.

Mobile phase A: 20 mmol/L Tris+5 mmol/L EDTA+2.0 mol/L NaCl, pH 8.5; and mobile phase B: 20 mmol/L Tris+5 mmol/L EDTA, pH 8.5.

Ammonium sulfate precipitation treatment: an appropriate amount of ammonium sulfate was weighed based on that the saturation concentration of ammonium sulfate should be 80%, added into the hydrophobic chromatography eluent, and stirred until the ammonium sulfate was completely dissolved. The mixture was placed in a refrigerator at 2-8° C., and continued stirring for 24 h, with a stirring speed of 100 rpm. The precipitate was isolated by centrifugation at 12000 rpm for 30 min at 4° C.

Dialysis treatment: 50 mM Tris-HCl buffer (pH 8.5) was taken to dissolve the precipitate in a ratio of the precipitate to the buffer of 1:10 (w/v, g/ml), and then the mixture was transferred to a dialysis bag (molecular weight cut-off: 100 kDa), stirred (100 rpm) together with 20 volumes of 50 mM Tris-HCl buffer (containing 250 mM NaCl, pH 8.5) to be dialyzed for 3 h at 4° ° C. The dialysate was then dialyzed for 24 h under stirring (100 rpm) together with 50 mM Tris-HCl buffer (pH 8.5) to be dialyzed in a volume 20 times that of the dialysate.

DEAE anion chromatography: the above dialysate was directly loaded for chromatography. Composition of mobile phase A: 20 mmol/L Tris, pH 8.5. Composition of mobile phase B: 20 mmol/L Tris+1.0 mol/L NaCl, pH 8.5. The steps of chromatography: the mobile phase A was equilibrated 3 CV, and loaded to the loading amount; the mobile phase A2 was washed 3 CV, and 0%-50% of the mobile phase B was eluted in a gradient mode 20 CV, thereby obtaining the eluent.

G-25M molecular exclusion chromatography: the eluent obtained from the DEAE anion chromatography was directly loaded for the molecular exclusion chromatography treatment. Mobile phase: 20 mmol/L Tris, pH 8.5, loading amount≤30% column volume/cycle, linear flow rate 300 cm/h. Each eluted peak was collected. The combined sample was subjected to SDS-PAGE electrophoresis for purity detection to obtain BoNT/A mutant 1.

### Example 8: Structure Identification of BoNT/A Mutant 1

### 1. Complete Molecular Weight Detection

Sample processing: 1 ml of the BoNT/A mutant 1 sample prepared in Example 7 was concentrated 5-fold and mixed evenly for loading.

### UPLC Conditions

Chromatographic column: BioResolve RP mAb 2.7 μm, 2.1 mm×100 mm, Waters 01093809916819; column temperature: 50° C.; detection wavelength: 280 nm; flow rate: 0.3 ml/min; loading amount: 10 μl.

Gradient:

| Time (min) | 0 | 12 | 13 | 15 | 16 | 20 |
|---|---|---|---|---|---|---|
| B | 10 | 60 | 90 | 90 | 10 | 10 |

MS conditions: ionization mode: ESI positive; mass scan range: 300-4000 Da; capillary voltage: 3.0 KV; source temperature: 100° C.; cone voltage: 150 KV; de-solvation gas temperature: 450° C.; cone blowback gas flow rate: 50 L/H; de-solvation gas flow rate: 800 L/H.

Data collection and processing: data collection was performed using Masslynx V4.1 software and data analysis was performed by means of UNIFI software. The complete molecular weight data analysis can refer to Table 8.

TABLE 8
Complete molecular weight data analysis for BoNT/A mutant 1

| | | Theoretical | Measured | | |
|---|---|---|---|---|---|
| | | molecular | molecular | | |
| | Identified | weight | weight | Error | |
| Peak | component | (Da) | (Da) | (ppm) | Proportion |
| 1 | BONT/A | 149216.3987 | 149218.3271 | 12.9 | 99.3% |
| | mutant | | | | |
| 2 | BoNT/A | 149218.4025 | 149220.3026 | 12.7 | 0.7% |
| | mutant | | | | |
| | (deamidated | | | | |
| | modification) | | | | |

### 2. Reduced Molecular Weight Detection

Sample processing: 150 μl of the BoNT/A mutant 1 sample prepared in Example 7 was taken and added with 150 μl of 7 mol/L guanidine hydrochloride/0.1 mol/L Tris (pH=8.0) and 3 μl of 1 mol/L DTT, incubated at 70° ° C. for 30 min and mixed evenly.

UPLC conditions: chromatographic column: BioResolve RP mAb 2.7 μm, 2.1 mm×100 mm, Waters 01093809916819; column temperature: 50° C.; detection wavelength: 280 nm; flow rate: 0.3 ml/min; and loading amount: 10 μl.

Gradient:

| Time (min) | 0 | 12 | 13 | 15 | 16 | 20 |
|---|---|---|---|---|---|---|
| B | 10 | 60 | 90 | 90 | 10 | 10 |

MS conditions: ionization mode: ESI positive; mass scan range: 300 to 4000 Da; capillary voltage: 3.0 KV; source temperature: 100° C.; cone voltage: 40 KV; de-solvation gas temperature: 450° C.; cone blowback gas flow rate: 50 L/H; and de-solvation gas flow rate: 800 L/H;

Data collection and processing: data collection was performed using Masslynx V4.1 software and data analysis was performed by means of UNIFI software. The reduced molecular weight data analysis can refer to Table 9.

TABLE 9
Reduced molecular weight data analysis of BoNT/A mutant 1

| | | Theoretical | Measured | |
|---|---|---|---|---|
| | Identified | molecular weight | molecular weight | Error |
| Peak | component | (Da) | (Da) | (ppm) |
| 1 | Light chain | 51107.6307 | 51108.1112 | 9.4 |
| | mutant | | | |
| 2 | Heavy chain | 98113.7816 | 98115.5619 | 18.1 |
| | mutant | | | |

### 3. Disulfide Bond Analysis

Sample processing: 1 ml of the BoNT/A mutant 1 sample prepared in Example 7 was taken and subjected to 5-fold concentration. 350 μl of 0.05 mol/L ammonium bicarbonate was added into each concentration tube, mixed evenly, and concentrated to 100 μl. 4 μl of 1 mol/L iodoacetamide solution (IAM) and 350 μl of 0.05 mol/L ammonium bicarbonate were added into each concentration tube, mixed evenly, and concentrated to 100 μl. 180 μl of the concentrated sample was taken and added with 20 μl of 1% RapiGest SF, incubated at constant temperature of 60° C. for 30 min, added with 8 μg of trypsin, incubated at 37° C. overnight, taken out, added with 1 μl of formic acid, incubated at 37° C. for 45 min, taken out, and centrifuged for 10 min at 13000 rpm. The supernatant was mixed for loading.

UPLC conditions: chromatographic column: UPLC BEH C18 1.7 μm, 2.1 mm×150 mm, Waters 01443804318321; column temperature: 60° C.; detection wavelength: 215 nm; flow rate: 0.3 ml/min; and loading amount: 10 μl.

Gradient:

| Time (min) | 0 | 5 | 140 | 141 | 145 | 146 | 150 |
|---|---|---|---|---|---|---|---|
| B | 2 | 2 | 40 | 100 | 100 | 2 | 2 |

MS conditions: ionization mode: ESI positive; mass scan range: 100-2000 Da; capillary voltage: 3.0 KV; source temperature: 100° C.; cone voltage: 40 KV; de-solvation gas temperature: 450° C.; cone blowback gas flow rate: 50 L/H; and de-solvation gas flow rate: 800 L/H.

Structural identification of wild-type BoNT/A: the contents and methods of structure identification were the same as those in Example 3 (preparation of the wild-type BoNT/A sample, see Examples 1 and 2 for the specific preparation methods).

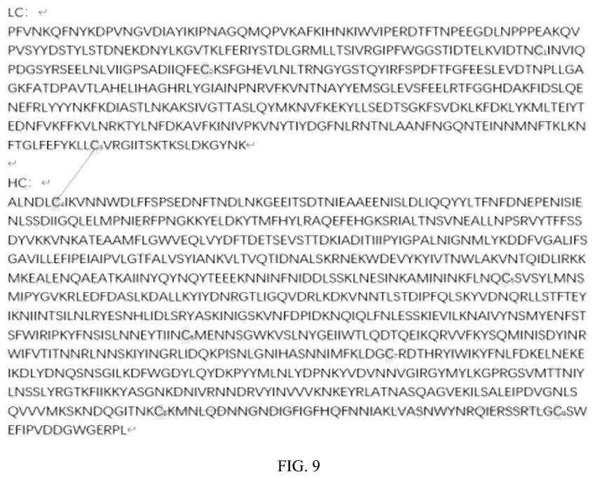

Data collection and processing: data collection was performed using Masslynx V4.1 software and data analysis was performed by means of UNIFI software. The specific results can refer to Table 10 and Table 11. Table 10 shows the detection results of wild-type BoNT/A sample, and Table 11 shows the detection results of BoNT/A mutant 1 sample prepared in Example 7. The positions of C₁-C₉ are specifically shown in FIG. 9, where Cn represents the n-th cysteine in the wild-type BoNT/A sequence from the N-terminus to the C-terminus; C₁ is the first cysteine; C₂ is the second cysteine; and Co is the ninth cysteine.

TABLE 10
Analysis of disulfide bonds in wild-type botulinum toxin A

| | Disulfide bond | Total signal | Total |
|---|---|---|---|
| | linkage | response value | percent |
| Disulfide bond | C₃═C₄ | 512932919 | 94.80% |
| correctly linked | C₈═C₉ | | |
| Disulfide bond | C₁═C₂ | 28133240 | 5.20% |
| mismatched | C₁═C₃ | | |
| | C₁═C₈ | | |
| | C₂═C₃ | | |
| | C₂═C₈ | | |
| | C₂═C₉ | | |
| | C₃═C₅ | | |
| | C₃═C₆ | | |
| | C₃═C₇ | | |
| | C₃═C₈ | | |
| | C₃═C₉ | | |
| | C₄═C₈ | | |
| | C₄═C₉ | | |
| | C₅═C₇ | | |
| | C₅═C₈ | | |
| | C₆═C₈ | | |
| | C₇═C₈ | | |
| | C₇═C₉ | | |

TABLE 11
Analysis of disulfide bonds in BoNT/A mutant 1

| | Disulfide bond | Total signal | Total |
|---|---|---|---|
| | linkage | response value | percent |
| Disulfide bond | C₃═C₄ | 619401425 | 100.00% |
| correctly linked | C₈═C₉ | | |
| Disulfide bond | C₃═C₈ | — | — |
| mismatched | C₃═C₉ | — | — |
| | C₄═C₈ | — | — |
| | C₄═C₉ | — | — |

It can be seen from the above that a rate of correct disulfide linkage in BoNT/A mutant 1 was 100.00%, and no disulfide bond mismatch was found. In contrast, a rate of correct disulfide linkage in wild-type BoNT/A was 94.80%, and 5.20% mismatch occurred, in which the mismatch rate of C₃=C₈ was 0.67%, and the mismatch rate of C₃=C₉ was 0.23%. Thus, the rate of disulfide bond mismatch in the BoNT/A mutant 1 according to the present disclosure is significantly reduced, without the mismatches of C₃=C₈ and C₃=C₉.

### Example 9: Preparation of BoNT/A Mutant 2 and Identification of Disulfide Bond Structure

For the preparation of BoNT/A mutant 2 (referred to as mutant 2 for short), the specific preparation method can refer to Example 6 and Example 7, and the content and method of structural identification can refer to Example 8, and the difference only lies in the amino acid sequence of mutant 2 (compared with the wild-type BoNT/A, the mutation positions of mutant 2 are C134G, C165G, C791A, C967A, and C1060 G). Accordingly, for the mutant 2, the amino acid sequence of the light chain mutant is set forth in SEQ ID NO: 5, and the nucleotide sequence of the light chain mutant of mutant 2 is set forth in SEQ ID NO: 11; and the amino acid sequence of the heavy chain mutant is set forth in SEQ ID NO: 6, and the nucleotide sequence of the heavy chain mutant is set forth in SEQ ID NO: 12. The disulfide bond in the BoNT/A mutant 2 was tested as described in Example 8 and the data are shown in Table 12.

TABLE 12
Analysis of disulfide bond BoNT/A mutant 2

| | Disulfide bond | Total signal | Total |
|---|---|---|---|
| | linkage | response value | percent |
| Disulfide bond | C₃═C₄ | 583222471 | 99.71% |
| correctly linked | C₈═C₉ | | |
| Disulfide bond | C₃═C₈ | 1111346 | 0.19% |
| mismatched | C₃═C₉ | 584919 | 0.10% |
| | C₄═C₈ | — | — |
| | C₄═C₉ | — | — |

The nucleotide sequence encoding the light chain mutant of the BoNT/A mutant 2 is set forth in SEQ ID NO: 11:

(SEQ ID NO: 11)
ATGCCGTTTGTGAACAAACAGTTTAACTATAAAGATCCGGTGAAC
GGCGTGGATATTGCGTATATTAAAATTCCGAACGCGGGTCAGATG
CAGCCGGTGAAAGCGTTTAAAATTCATAACAAAATTTGGGTGATT
CCGGAACGCGATACCTTTACCAACCCGGAAGAAGGCGATCTGAAC
CCGCCACCGGAAGCGAAACAAGTGCCGGTGAGCTATTATGATAGC
ACCTATCTGAGCACCGATAACGAAAAAGATAACTATCTGAAAGGC
GTGACCAAACTGTTTGAACGCATTTATAGCACCGATCTGGGCCGC
ATGCTGCTGACGAGCATTGTGCGCGGCATTCCGTTCTGGGGCGGC
AGCACCATTGATACCGAACTGAAAGTGATTGATACCAACGGTATT
AACGTGATTCAGCCGGATGGCAGCTATCGCAGCGAAGAACTGAAC
CTGGTGATTATTGGCCCGAGCGCGGATATTATTCAGTTTGAAGGT
AAAAGCTTTGGCCATGAAGTGCTGAACCTGACCCGCAACGGCTAT
GGCAGCACGCAGTATATTCGCTTTAGCCCGGATTTTACCTTTGGC
TTTGAAGAAAGCCTGGAAGTGGATACCAACCCGCTGCTGGGCGCG
GGCAAATTTGCGACCGATCCGGCGGTGACCCTGGCGCATGAACTG
ATTCATGCGGGCCATCGCCTGTATGGCATTGCGATTAACCCGAAC
CGCGTGTTTAAAGTGAACACCAACGCGTATTATGAAATGAGCGGC
CTGGAAGTGAGCTTTGAAGAACTGCGCACCTTTGGCGGCCATGAT
GCGAAATTTATTGATAGCCTGCAAGAAAACGAATTTCGCCTGTAT
TACTATAACAAATTTAAAGATATTGCGAGCACCCTGAACAAAGCG
AAAAGCATTGTGGGCACCACCGCGAGCCTGCAGTATATGAAAAAC
GTGTTTAAAGAAAAATATCTGCTGAGCGAAGATACGAGCGGCAAA
TTTAGCGTGGATAAACTGAAATTTGATAAACTGTATAAAATGCTG
ACCGAAATTTATACCGAAGATAACTTTGTGAAATTTTTTAAAGTG
CTGAACCGCAAGACCTATCTGAACTTTGATAAAGCGGTGTTTAAA
ATTAACATTGTGCCGAAAGTGAACTATACCATTTATGATGGCTTT
AACCTGCGCAACACCAACCTGGCGGCGAACTTTAACGGTCAGAAC
ACCGAAATTAACAACATGAACTTTACCAAACTGAAAAACTTTACC
GGCCTGTTTGAATTTTATAAACTGCTGTGCGTTCGCGGCATCATT
ACGAGCAAAACCAAAAGCCTGGATAAAGGCTATAACAAATAA.

The nucleotide sequence encoding the heavy chain mutant of the BoNT/A mutant 2 is set forth in SEQ ID NO: 12:

(SEQ ID NO: 12)
ATGGCGCTGAACGATCTGTGCATTAAAGTGAATAATTGGGATCTG
TTTTTTAGCCCGAGCGAAGATAACTTTACCAACGATCTGAACAAA
GGCGAAGAAATTACGAGCGATACCAACATTGAAGCGGCGGAAGAG
AACATTAGTCTGGATCTGATTCAGCAGTATTATCTGACCTTTAAC
TTTGATAACGAACCGGAAAACATTAGTATTGAAAACCTGAGCAGC
GATATTATTGGTCAGCTGGAACTGATGCCGAACATTGAACGCTTT
CCGAACGGCAAAAAATATGAACTGGATAAATATACCATGTTTCAT
TATCTGCGCGCGCAAGAATTTGAACATGGCAAAAGCCGCATTGCG
CTGACCAACAGCGTGAACGAAGCGCTGCTGAACCCGAGCCGCGTG
TATACCTTTTTTAGCAGCGATTATGTGAAAAAAGTGAACAAAGCG
ACCGAAGCGGCGATGTTTCTGGGCTGGGTGGAACAGCTGGTGTAT
GATTTTACCGATGAGACGAGCGAAGTGAGTACCACCGATAAAATT
GCGGATATTACCATTATCATTCCGTATATTGGCCCGGCGCTGAAC
ATTGGCAACATGCTGTATAAAGATGATTTTGTGGGCGCGCTGATT
TTTAGCGGCGCGGTGATTCTGCTGGAATTTATTCCGGAAATCGCG
ATTCCGGTGCTGGGCACCTTTGCGCTGGTGAGCTATATTGCGAAC
AAAGTGCTGACCGTGCAGACCATTGATAACGCGCTGAGCAAACGC
AACGAAAAATGGGATGAAGTGTATAAATATATTGTGACCAACTGG
CTGGCGAAAGTGAACACGCAGATTGATCTGATTCGCAAAAAAATG
AAAGAAGCGCTGGAAAACCAAGCGGAAGCGACCAAGGCGATTATT
AACTATCAGTATAATCAGTATACCGAAGAGGAAAAAAACAACATT
AACTTTAACATTGATGATCTGAGCAGCAAATTAAATGAAAGCATT
AACAAAGCGATGATCAACATTAACAAGTTTCTGAATCAGGCAAGC
GTGAGCTATCTGATGAACAGCATGATTCCGTATGGCGTGAAACGC
CTGGAAGATTTTGATGCGAGCCTGAAAGATGCGCTGCTGAAATAT
ATTTATGATAACCGCGGCACCCTGATTGGCCAAGTGGATCGCCTG
AAAGATAAAGTTAATAACACGCTGAGCACCGATATTCCGTTTCAG
CTGAGCAAATATGTGGATAATCAGCGCCTGCTGAGCACCTTTACC
GAATATATTAAAAACATTATTAACACGAGCATTCTGAACCTGCGC
TATGAAAGCAACCATCTGATTGATCTGAGCCGCTATGCGAGCAAA
ATTAACATTGGCAGCAAAGTGAACTTTGATCCGATTGATAAAAAT
CAGATTCAGCTGTTTAACCTGGAAAGCAGCAAAATTGAAGTGATT
CTGAAAAACGCGATTGTGTATAACAGCATGTATGAAAACTTTAGC
ACGAGCTTTTGGATTCGCATTCCGAAATACTTTAACAGCATCAGC
CTGAACAACGAATATACCATTATTAACGCAATGGAAAACAACAGC
GGCTGGAAAGTGAGCCTGAACTATGGCGAAATTATTTGGACCCTG
CAAGATACCCAAGAAATTAAACAGCGCGTGGTGTTTAAATATAGT
CAGATGATTAACATTAGCGATTATATTAACCGCTGGATTTTTGTG
ACCATTACCAACAACCGTCTGAACAACAGCAAAATTTATATTAAC
GGCCGCCTGATTGATCAGAAACCGATTAGCAACCTGGGCAACATT
CATGCGAGCAACAACATTATGTTTAAACTGGATGGCGGTCGCGAT
ACGCATCGCTATATCTGGATTAAATATTTTAATCTGTTCGACAAA
GAACTGAACGAAAAAGAAATTAAAGATCTGTATGATAATCAGAGC
AACAGCGGCATTCTGAAAGATTTTTGGGGCGATTATCTGCAGTAT
GATAAACCGTATTATATGCTGAACCTGTATGATCCGAACAAATAT
GTGGATGTGAACAACGTGGGCATTCGCGGCTATATGTATCTGAAA
GGCCCGCGCGGCAGCGTGATGACCACCAACATTTATCTGAACAGC
AGCCTGTATCGCGGCACCAAATTTATTATTAAAAAATATGCGAGC
GGCAACAAAGATAACATTGTGCGCAACAACGATCGCGTGTATATT
AACGTGGTTGTGAAAAACAAAGAATATCGCCTGGCGACCAACGCG
AGCCAAGCGGGCGTGGAAAAAATTCTGAGCGCGCTGGAAATTCCG
GATGTGGGCAACCTGAGCCAAGTGGTTGTGATGAAAAGCAAAAAC
GATCAAGGCATTACCAACAAGTGCAAAATGAACCTGCAAGATAAC
AACGGCAACGATATTGGCTTTATTGGCTTTCATCAGTTTAACAAC
ATTGCGAAACTGGTGGCGAGCAACTGGTATAACCGTCAGATTGAA
CGCAGCAGCCGCACCCTGGGCTGCAGCTGGGAATTTATTCCGGTT
GATGATGGCTGGGGCGAACGCCCGCTGTAA.

### Example 10: Determination of Activity of BoNT/A Mutant Protein

Test sample: BoNT/A mutant 1 prepared in Example 7, BoNT/A mutant 2 prepared in Example 9, and the wild-type BoNT/A prepared in Example 2 as set forth in SEQ ID NO: 6.

Experimental design and grouping administration: at the end of adaptive feeding of 162 mice, 54 mice were respectively administrated with 3 test samples. After administration, the toxicity reaction of mice and the death of animals in each group were closely observed for 4 consecutive days.

Before the experiment, the experimental mice were weighed and distributed to each group according to the average body weight, ensuring that there was no statistical difference in the average body weight of animals in each group. Each experiment was divided into 9 groups, with 6 animals in each group, half males and half females. The test sample was administered through intraperitoneal injection. One person took the test solution, and another person checked; one person held the animal, and another person completed the animal administration. The administration time was recorded after each injection. The grouping of the test sample, BoNT/A mutant 1, and the dosing information are shown in Table 13.

TABLE 13
statistical table of mice death record of test sample BoNT/A mutant 1

| | | | | Survival | Cumulative | Cumulative | Cumulative |
|---|---|---|---|---|---|---|---|
| Dosing | Dosing | Animal | Total death | animal | death | survivor | mortality |
| (pg/animal) | logarithm | number | number | Number | number | number | (%) |
| 17.85714 | 1.25181 | 6 | 6 | 0 | 49 | 0 | 100 |
| 12.75510 | 1.10568 | 6 | 6 | 0 | 43 | 0 | 100 |
| 9.11079 | 0.95956 | 6 | 6 | 0 | 37 | 0 | 100 |
| 6.50771 | 0.81343 | 6 | 6 | 0 | 31 | 0 | 100 |
| 4.64836 | 0.66730 | 6 | 6 | 0 | 25 | 0 | 100 |
| 3.32026 | 0.52117 | 6 | 5 | 1 | 19 | 1 | 93.75 |
| 2.37161 | 0.37504 | 6 | 5 | 1 | 14 | 2 | 83.33333 |
| 1.69401 | 0.22892 | 6 | 2 | 4 | 5 | 6 | 45.45455 |
| 1.21001 | 0.08279 | 6 | 3 | 3 | 3 | 9 | 25 |

As shown in Table 13 above, 50% cumulative mortality rate is between 45.45455% and 83.33333%.

Under the experimental conditions, the LD₅₀ of the test sample, BoNT/A mutant 1, was calculated to be 1.76381 pg/animal based on Reed-Muench method, and the converted sample virulence was 5.67×10⁸ LD₅₀/mg.

Under the same experimental conditions, the LD₅₀ of the test sample, BoNT/A mutant 2 was calculated to be 3.0770 pg/animal based on Reed-Muench method, and the converted sample virulence was 3.25×10⁸ LD₅₀/mg.

Under the same experimental conditions, the LD₅₀ of the test sample, the wild-type BoNT/A was calculated to be 4.5662 pg/animal based on Reed-Muench method, and the converted sample virulence was 2.19×10⁸ LD₅₀/mg.

Comparison of virulence of 3 different test samples is shown in Table 14.

TABLE 14
Comparison of virulence of test samples, i.e., BoNT/A
mutant 1, BoNT/A mutant 2, and wild type BoNT/A

| Test sample (Lot #) | LD₅₀ (pg/animal) | Virulence (LD₅₀/mg) |
|---|---|---|
| Wild type BoNT/A | 4.5662 | 2.19 ± 10⁸ |
| BoNT/A mutant 1 | 1.7638 | 5.67 ± 10⁸ |
| BoNT/A mutant 2 | 3.0770 | 3.25 ± 10⁸ |

As revealed in Table 14, the BoNT/A mutant 1 according to the present disclosure has a higher biological activity (virulence) than the wild-type BoNT/A, having a virulence 2.6 times that of the wild-type BoNT/A.

While the embodiments of the present disclosure have been illustrated and described above, it will be understood that the above-described embodiments are illustrative, rather than limiting the present disclosure. Those skilled in the art can make changes, modifications, substitutions, and alterations without departing from the scope of the present disclosure.

What is claimed is:

1. A botulinum neurotoxin of type A (BoNT/A) mutant, comprising:
a first peptide fragment; and
a second peptide fragment, the first peptide fragment and the second peptide fragment being linked through an interchain disulfide bond,
wherein the first peptide fragment has a mutation at position 134 and/or position 165 compared to a light chain of a wild-type BoNT/A; and/or
wherein the second peptide fragment has at least one of the following mutation positions compared to a heavy chain of the wild-type BoNT/A: positions 791, 967, and 1060.

2. The BoNT/A mutant according to claim 1, characterized in that, the interchain disulfide bond is formed by a cysteine at position 430 in the first peptide fragment and a cysteine at position 454 in the second peptide fragment.

3. The BoNT/A mutant according to claim 1, characterized in that,
the first peptide fragment has mutations at positions 134 and 165 compared to the light chain of the wild-type BoNT/A; and/or
the second peptide fragment has mutations at positions 791, 967 and 1060 compared to the heavy chain of the wild-type BoNT/A.

4. The BoNT/A mutant according to claim 1, characterized in that, cysteine at position 134, 165, 791, 967 or 1060 is mutated to one of the following amino acids: G, A, S, E, and P.

5. The BoNT/A mutant according to claim 1, characterized in that, C at position 134 in the first peptide fragment is mutated to G, A, or S.

6. The BoNT/A mutant according to claim 1, characterized in that, C at position 165 in the first peptide fragment is mutated to G, A, P, or S.

7. The BoNT/A mutant according to claim 1, characterized in that, C at position 791 in the second peptide fragment is mutated to G, A, or S.

8. The BoNT/A mutant according to claim 1, characterized in that, C at position 967 in the second peptide fragment is mutated to G, A, or S.

9. The BoNT/A mutant according to claim 1, characterized in that, C at position 1060 in the second peptide fragment is mutated to G, A, E, or S.

10. The BoNT/A mutant according to claim 1, characterized in that, the first peptide fragment has mutations C134G and C165P compared to the light chain of the wild-type BoNT/A.

11. The BoNT/A mutant according to claim 1, characterized in that, the second peptide fragment has mutations C791A, C967A, and C1060E compared to the heavy chain of the wild-type BoNT/A.

12. The BoNT/A mutant according to claim 1, characterized in that,
the first peptide fragment has an amino acid sequence as set forth in SEQ ID NO: 3 or 5; or
the second peptide fragment has an amino acid sequence as set forth in SEQ ID NO: 4 or 6.

13. The BoNT/A mutant according to claim 1, characterized in that,
the first peptide fragment of the BoNT/A mutant has an amino acid sequence as set forth in SEQ ID NO: 3, and the second peptide fragment of the BoNT/A mutant has an amino acid sequence as set forth in SEQ ID NO: 4; or
the first peptide fragment of the BoNT/A mutant has an amino acid sequence as set forth in SEQ ID NO: 5, and the second peptide fragment of the BoNT/A mutant an amino acid sequence as set forth in SEQ ID NO: 6.

14. A nucleic acid molecule, encoding the first peptide fragment and/or the second peptide fragment of the BoNT/A mutant according to claim 1.

15. An expression vector, carrying the nucleic acid molecule according to claim 14.

16. Genetic engineering bacteria, comprising:
genetic engineering bacteria carrying the nucleic acid molecule according to claim 14.

17. A pharmaceutical composition, comprising the BoNT/A mutant according to claim 1.

18. The pharmaceutical composition according to claim 17, characterized by further comprising a pharmaceutically acceptable adjuvant, wherein the pharmaceutically acceptable adjuvant comprises at least one selected from a buffer, a protective agent, an active agent, and an excipient.

19. A method for ameliorating and/or treating a disease, comprising:
administrating to a subject the BoNT/A mutant according to claim 1,
wherein the disease comprises at least one of strabismus, cervical dystonia, laryngeal dystonia, upper limb focal dystonia, primary hand tremor, sialorrhea, blepharospasm, hemifacial spasm, stroke-caused upper/lower limb spasm, cerebral palsy-caused upper/lower limb spasm, axillary hyperhidrosis, palmar hyperhidrosis, detrusor-sphincter dyssynergia, chronic migraine, and neurogenic and idiopathic overactive bladder.

20. A method for medical cosmetology, comprising:
administrating to a subject the BoNT/A mutant according to claim 1, wherein the medical cosmetology comprises wrinkle removal and/or facial slimming, or the medical cosmetology comprises amelioration and/or treatment of at least one of the following symptoms:
frown lines, crow's feet, and forehead wrinkles.
